(19) United States
(12) Patent Application Publication
(10) Pub. No.: US 2024/0350519 A1
(43) Pub. Date: Oct. 24, 2024

(54) MICROEMULSION DELIVERY SYSTEMS INCORPORATED INTO GUMMY CONFECTIONS

(71) Applicant: Quicksilver Scientific, Inc., Louisville, CO (US)

(72) Inventors: Christopher W. Shade, Louisville, CO (US); Steven Tieu, Louisville, CO (US); James R Huey, Denver, CO (US); David Durkee, Louisville, CO (US); Michelle Schwenk, Louisville, CO (US)

(73) Assignee: Quicksilver Scientific, Inc., Louisville, CO (US)

(21) Appl. No.: 18/615,236

(22) Filed: Mar. 25, 2024

Related U.S. Application Data
(63) Continuation of application No. PCT/US22/46032, filed on Oct. 7, 2022.
(60) Provisional application No. 63/253,207, filed on Oct. 7, 2021.

(51) Int. Cl. A61K 31/00; A23G 3/34; A23G 3/36; A23G 3/40; A23G 3/42; A23G 3/44; A23G 3/48; A61K 9/00; A61K 9/107; A61K 31/01; A61K 31/015; A61K 31/045; A61K 31/07; A61K 31/122; A61K 31/197; A61K 31/198; A61K 31/205; A61K 31/355; A61K 31/375; A61K 31/593; A61K 33/30; A61K 36/185; A61K 36/539; A61K 47/10; A61K 47/12; A61K 47/14; A61K 47/24; A61K 47/26; A61K 47/42; A61K 47/44

(52) U.S. Cl. CPC A61K 31/658; A23G 3/0019; A23G 3/368; A23G 3/40; A23G 3/42; A23G 3/44; A23G 3/48; A61K 9/0056; A61K 9/1075; A61K 31/01; A61K 31/015; A61K 31/045; A61K 31/07; A61K 31/122; A61K 31/197; A61K 31/198; A61K 31/205; A61K 31/355; A61K 31/375; A61K 31/593; A61K 33/30; A61K 36/185; A61K 36/539; A61K 47/10; A61K 47/12; A61K 47/14; A61K 47/24; A61K 47/26; A61K 47/42; A61K 47/44

(57) ABSTRACT

Microemulsions incorporated into gummy confections are described where hydrophobic liquid droplets are distributed in a continuous hydrophilic liquid phase. In relation to conventional oil-in-water (OIW) microemulsions, prior to incorporation into the gummy confections the described microemulsions may be thought of as modified oil-in-water (MOIW) microemulsions, where both the “oil” and “water” phases of the microemulsion are modified. The oil phase droplets of the MOIW microemulsion are modified with alcohol and can better deliver oil-soluble species to the bloodstream than can oil blends or the oil phases of conventional oil-in-water (OIW) microemulsions. The polar continuous “water” phase of the MOIW microemulsion is modified with a sugar or sugar alcohol. The modified oil phase droplets disperse into the modified polar continuous phase of the MOIW microemulsion prior to incorporation into the gummy confection.

### REFERENCE TO RELATED APPLICATIONS

This application is a continuation of International Application No. PCT/US2022/046032, filed Oct. 7, 2022, entitled “Microemulsion Delivery Systems Incorporated into Gummy Confections”, which claims the benefit of U.S. Provisional Application No. 63/253,207 entitled “Microemulsion Delivery Systems Incorporated into Gummy Confections” filed Oct. 7, 2021, both of which are incorporated by reference in the entirety.

### BACKGROUND

Cannabinoids are compounds that act on the cannabinoid receptors in cells that alter neurotransmitter release. Cannabinoids include the endocannabinoids, which are produced naturally in the bodies of animals, phytocannabinoids, which are found in plants of the Cannabis genus and in some other plants, and synthetic cannabinoids that are synthesized. Type 1 cannabinoid receptors are found primarily in the brain and are absent from the part of the brain stem responsible for respiratory and cardiovascular function. Type 2 cannabinoid receptors are predominantly found in the immune system and appear to be responsible for the anti-inflammatory and possibly other therapeutic effects.

Phytocannabinoids are isolated from plants of the Cannabis genus, which is believed to include three species, Cannabis sativa, Cannabis indica, and Cannabis ruderalis. Cannabis plants including less than 0.3% tetrahydrocannabinol (THC) by weight are commonly referred to as “hemp”, while plants including 0.3% or greater by weight THC are commonly referred to as marijuana. At least 113 different phytocannabinoids may be isolated from plants of the Cannabis genus. The phytocannabinoids are isolated in their “A” or acidic form and are then decarboxylated, often by heat, to their more biologically active, decarboxylated forms.

THC is the most famous cannabinoid as it binds to the Type 1 receptors and is considered psychoactive. Cannabidiol (CBD) is becoming a more commonly known non-psychoactive cannabinoid as it acts on the Type 1 and Type 2 receptors and is known to reduce pain and inflammation and calm some nerve responses, such as those associated with Dravet syndrome in children. Additionally, CBD may counteract cognitive impairment associated with THC use, including short term memory loss and may have additional anti-psychotic effects in addition to serving as an antioxidant. Cannabigerol (CBG) is another non-psychoactive cannabinoid that may have similar effects to CBD. Cannabichromene (CBC), Cannabinol (CBN), and Cannabitriol (CBT) are other cannabinoids that are being studied for potential biological activity.

While work remains ongoing regarding the health benefits of cannabinoids, pharmacological utility has been demonstrated or is likely to be demonstrated for the previously mentioned Dravet syndrome in children, Parkinson's disease, schizophrenia, anxiety disorder, and inhibiting the development of some cancer cells. As the human endocannabinoid system is involved in basic life functions, including appetite, immune response, reproduction, and pain management, the effects of cannabinoids on the human body are likely diverse. The ability of cannabinoids to prevent the overactivation of these functions may provide a reduction in progression or prevention of diseases based on overactivation of these functions.

Terpenes are extracted from plants, such as conifers, flowers, citrus fruits, and some insects, such as termites and swallowtail butterflies. From a molecular perspective, all terpenes include isoprene functionality and are a diverse class of organic molecules. In addition to historical use as fragrances, terpenes provide a basis for biologically active substances including Vitamin-A and steroids. Terpenes include compounds such as limonene, pinene, linalool, and beta-caryophyllene. Beta-caryophyllene, for example, has use as a fragrance and as an anti-inflammatory.

Oral delivery of cannabinoids and terpenes with conventional delivery systems can result in negligible blood concentration after 20-minutes of administration and not provide an effective bloodstream concentration, which is believed to be approximately 0.4 ng/mL and higher in the bloodstream for cannabinoids. In fact, for low absorbing individuals, effective bloodstream concentrations may not be reached after 90-minutes of administration, or at all, with conventional oral delivery systems without consuming undesirably large quantities of the conventional oral delivery system. Hence of the cannabinoids or terpenes consumed with conventional oral delivery systems, a significant majority of that consumed may be excreted and never used.

Emulsions are mixtures of two or more liquids that do not solubilize. Thus, the two or more liquids do not form a solution and an identifiable interface exists between the combined liquids. Emulsions may be macroemulsions, pseudo-emulsions, nanoemulsions or microemulsions. Emulsions may be used for parenteral delivery, ocular delivery, transdermal delivery, oral delivery, and the like.

FIG. 1A represents an example nanoemulsion droplet 100 having a single wall of phospholipids (monolayer) forming a hydrophilic exterior 120 and a hydrophobic interior 110. The monolayer wall of the nanoemulsion droplet 100 is formed from a single layer of phospholipids. The outer wall 120 is water soluble due to the phosphate functionality while the interior 110 is fat-soluble due to the alkyl functionality. FIG. 1B represents multiple of the nanoemulsion droplets 100 in a continuous phase 150.

FIG. 2A represents a microemulsion droplet 200 having a single wall of phospholipids (monolayer) forming a hydrophilic exterior 220 and a hydrophobic interior 210. As with the nanoemulsion droplets 100, the monolayer wall of the microemulsion droplet 200 is formed from a single layer of phospholipids. In relation to the represented nanoemulsion droplets 100, the microemulsion droplets 200 are substantially smaller in diameter—which is often the case for microemulsions. In fact, the diameter of the microemulsion droplets 200 is reduced to where non-polar tails 230 of the monolayer phospholipids are “crushed” into each other, thus forming a more “solid” interior hydrophobic barrier than in the case of the nanoemulsion droplets 100 as represented in FIG. 1. FIG. 2B represents multiple microemulsion droplets 200 in a continuous phase 250. Also represented in the continuous phase 250 are a few individual phospholipid molecules 260 not incorporated into the microemulsion droplets 200.

Transdermal creams are typically “pseudo-emulsions” with solid granules of the deliverable not fully solubilized in the droplets of the emulsion forming the cream. In contrast to the larger droplet macro- and pseudo-emulsions, the smaller droplets of nanoemulsions and microemulsions provide the potential to provide better delivery performance than conventionally available from macro- and pseudo-emulsions for either transdermal or oral adsorption.

While the high-energy mixing, in the form of pressure (including shear forces), temperature, and combinations thereof, used to form nanoemulsions can provide the smaller droplets of a microemulsion, such nanoemulsions are not thermally stable, thus are not shelf-stable microemulsions, and are like a macroemulsion in that the components of the nanoemulsion eventually separate into immiscible polar and non-polar liquids. Thus, as represented in FIG. 1 and FIG. 2, nanoemulsion droplets tend to be larger than microemulsion droplets as the nanoemulsion droplets continually expand in diameter after formation until the agglomerating droplets separate from the continuous phase.

Conventionally, macroemulsions, nanoemulsions, and microemulsions have been used for either oil-soluble or water-soluble deliverables. Cannabis extracts and terpenes are oil-soluble, but are absorbed relatively slowly and inconsistently through the gut when solubilized in oil. However, conventional oil-in-water (OIW) emulsions including Cannabis extracts and some terpenes, regardless of emulsion form, generally form oil droplets that readily dissociate from the water phase of the emulsion with higher concentrations of Cannabis extracts in the oil droplets.

Such dissociation of the oil droplets from the water phase of conventional oil-in-water emulsions results in a significant loss in blood uptake rate and total blood delivery of the cannabinoids and terpenes when delivered by conventional oil-in-water emulsions. This is believed attributable to the fact that when the oil droplets of the emulsion dissociate from the water phase, the resulting dissociated emulsion becomes oil, water, and whatever remains of the emulsion, with the dissociated oil having the approximate delivery profile of oil alone, thus becoming slow and inconsistent. Thus, conventional oil-in-water emulsions including Cannabis extracts and some terpenes tend to suffer from similar bloodstream uptake disadvantages to oil only formulations as the oil phase has dissociated from the water phase by the time the conventional emulsion is consumed.

In addition to blood uptake, emulsion stability can also influence the degradation rate of the deliverable. Many deliverables are sensitive to the physical environment and lose their potency if proper hydration or pH is not maintained during storage. Thus, the efficacy of a deliverable may be significantly compromised by relatively small changes in the emulsion constituents.

In addition to liquid preparations, conventional “gummy” or “soft-chew” systems have been used to deliver actives when eaten. Gummies are generally gelatin- or pectin-based confections having an elasticity that is sufficient to substantially reconstitute the mass of the confection after each chew. Thus, gummy confections are elastic solids that do not readily separate during chewing, instead requiring significant chewing to separate the material into reduced size pieces. Gummy confections also have “memory” arising from the elasticity where when chewed the confection reforms something approximating its original shape. Together, these attributes provide a “gummy mouthfeel” or chew quality to the confection.

While the nature of the gummy confection results in an extended mouth residence time and can thus provide enhanced deliverable uptake in relation to a preparation that is initially swallowed after consumption, it can be difficult to incorporate an emulsion into a gummy confection due the specific water content required for gummy formation and incompatibility between the emulsion forming versus the gummy forming constituents. The ingredients that form the gummy may adversely interact with the structure of the emulsion or the reverse. Thus, while deliverability advantages may be obtained from incorporation into conventional gummies, conventional gummies lack the ability of emulsions to rapidly deliver a deliverable to the bloodstream of a properly configured emulsion.

As can be seen from the above description, there is an ongoing need for simple and efficient materials and methods for oral delivery systems for delivering cannabinoids and terpenes quickly and in higher concentration per consumed amount to the bloodstream. Conventional oil mixtures have traditionally been plagued with exceedingly slow, low, and inconsistent uptake attributed to the GI adsorption pathway. Conventional oil-in-water emulsion systems have traditionally had disadvantages including poor stability to cold and heat, particularly regarding maintaining the desired average droplet diameter in the emulsion, which is important for effective intra-oral delivery to the bloodstream, preventing phase separation of the oil and water components, and preventing dissociation of the deliverable and/or the oil from the emulsion. In addition to these disadvantages resulting in slow, poor, and inconsistent blood uptake of the deliverable, conventional emulsion systems also have the disadvantage of requiring too great a volume of the emulsion in relation to the mass or volume of the deliverable.

The microemulsions, gummy confections, and methods of the present invention overcome at least one of the disadvantages associated with conventional oral delivery systems by allowing the convenient, rapid, efficient, and reproducible oral delivery of cannabinoids, oil-soluble vitamins, oil-soluble carotenoids, and terpenes to the bloodstream.

### SUMMARY

In one aspect, the invention provides a gummy confection composition comprising a modified oil-in-water microemulsion including a modified oil phase, a modified polar continuous phase, and an oil-soluble species, where the oil-soluble species is solubilized in the modified oil phase, the modified oil phase including a phospholipid, a polyethylene glycol derivative, an oil, and an alcohol, and where the modified polar continuous phase includes a sugar or sugar alcohol and water; and a gummy base, where the gummy base includes solid sugar, liquid syrup, gelling agent, and an edible acid.

In another aspect, the invention provides a method of making a gummy confection composition comprising a modified oil-in-water microemulsion composition, the method including combining the phospholipid, the polyethylene glycol derivative, the oil, and the alcohol to form an alcohol-lipid mixture; combining a sugar or sugar alcohol and water to form a modified polar continuous phase; combining the oil-soluble species with the alcohol-lipid mixture and the modified polar continuous phase at atmospheric pressure to form the modified oil-in-water microemulsion; combining a first component of the solid sugar, the gelling agent, and additional water to form a first gummy base component; combining a second component of the solid sugar, the liquid syrup, and additional water to form a second gummy base component; combining the first gummy base component, the second gummy base component, the modified oil-in-water microemulsion, and enough edible acid to provide a pH from 3.3 to 3.5 to form a combination; transferring the combination to a mold; and forming the gummy confection composition in the mold upon cooling.

In another aspect, the invention provides a method of orally delivering an oil-soluble species to the bloodstream of a human subject with the gummy confection composition where the method comprises introducing the gummy confection composition to a human subject; and delivering the oil-soluble species to the bloodstream of the human subject, where within 30 to 50 minutes of the human subject beginning to chew and ingest the gummy confection composition, the gummy confection composition provides the human subject a blood concentration Cmax of the oil-soluble species

In another aspect, the invention provides a gummy confection composition including a modified oil-in-water microemulsion comprising a modified oil phase, a modified polar continuous phase, and an oil-soluble species, where the oil-soluble species is solubilized in the modified oil phase, the modified oil phase comprising a phospholipid, a polyethylene glycol derivative, an oil, and an alcohol, and where the modified polar continuous phase comprises a sugar or sugar alcohol and water; and a gummy base, where the gummy base includes solid sugar, liquid syrup, gelling agent, an edible acid, and gummy base water.

In another aspect, the invention provides a method of making a gummy confection composition comprising a modified oil-in-water microemulsion composition, the method including combining a phospholipid, a polyethylene glycol derivative, an oil, and an alcohol to form an alcohol-lipid mixture; combining a sugar or sugar alcohol and water to form a modified polar continuous phase; combining an oil-soluble species with the alcohol-lipid mixture and the modified polar continuous phase at atmospheric pressure to form the modified oil-in-water microemulsion; combining a first component of a solid sugar, a gelling agent, and additional water to form a first a first gummy base component; combining a second component of the solid sugar, a liquid syrup, and additional water to form a second gummy base component; combining the first gummy base component, the second gummy base component, and the modified oil-in-water microemulsion to form a combination; transferring the combination to a mold; and forming the gummy confection composition in the mold upon cooling.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the claims that follow.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1A represents a nanoemulsion droplet having a single wall of phospholipids (monolayer) forming a hydrophilic exterior and a hydrophobic interior.

FIG. 1B represents multiple of the nanoemulsion droplets in a continuous phase.

FIG. 2A represents a microemulsion droplet having a single wall of phospholipids (monolayer) forming a hydrophilic exterior and a hydrophobic interior.

FIG. 2B represents multiple microemulsion droplets represented in a continuous phase.

FIG. 3 represents a method of making a MOIW microemulsion incorporated into a gummy confection, where the MOIW microemulsion includes an oil-soluble species.

FIG. 4 provides the results of a CBD blood uptake rate and concentration analysis in graphical form for liquid carrier systems.

FIG. 5 provides the cumulative Area Under the Curve (AUC) values in ug*(min/mL) determined from a bloodstream concentration analysis in graphical form for the liquid carrier systems.

FIG. 6 provides the results of a CBD blood uptake rate and concentration analysis in graphical form for the gummy confections and an oil blend.

FIG. 7 provides the cumulative Area Under the Curve (AUC) values in ug*(min/mL) determined from a bloodstream concentration analysis in graphical form for the gummy confections and the oil blend.

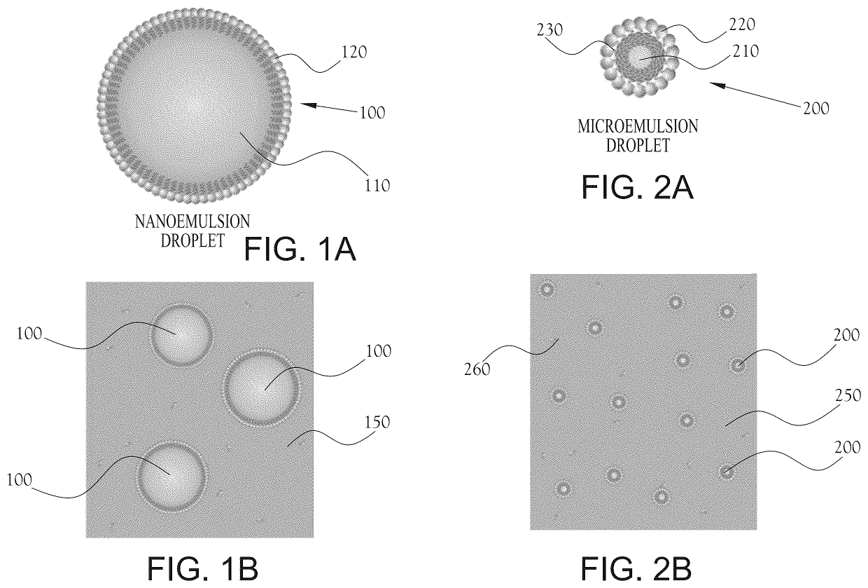

### DETAILED DESCRIPTION

Microemulsions incorporated into gummy confections are described where hydrophobic liquid droplets are distributed in a continuous hydrophilic liquid phase. In relation to conventional oil-in-water (OIW) microemulsions, prior to incorporation into the gummy confections the described microemulsions may be thought of as modified oil-in-water (MOIW) microemulsions, where both the “oil” and “water” phases of the microemulsion are modified. The oil phase droplets of the MOIW microemulsion are modified with alcohol and can better deliver oil-soluble species to the bloodstream than can oil blends or the oil phases of conventional oil-in-water (OIW) microemulsions. The polar continuous “water” phase of the MOIW microemulsion is modified with a sugar or sugar alcohol. The modified oil phase droplets disperse into the modified polar continuous phase of the MOIW microemulsion prior to incorporation into the gummy confection.

The MOIW microemulsion is configured to be incorporated into a gummy confection and upon release into the aqueous environment of the mouth from the gummy confection during chewing unexpectedly retains the superior bloodstream delivery of the oil-soluble species provided by the MOIW microemulsion in relation to conventional gummy confections. While not wishing to be bound by any particular theory, it is believed that due to the thermodynamic stability of the modified oil phase droplets and the sugar or sugar alcohol constituents included in the gummy confection, that the MOIW microemulsion is reconstituted as the gummy confection is chewed.

The MOIW microemulsion gummy confections can provide the uptake of the oil-soluble species to the bloodstream through the oral and gastric mucosa. The MOIW microemulsion gummy confections can orally deliver effective bloodstream concentrations of the oil-soluble species to the bloodstream of an individual faster, such as within 20-minutes of introduction, than conventional non-MOIW microemulsion gummy confections, which were unable to provide an approximate 4 ng/mL bloodstream concentration of the oil-soluble species until after 50-minutes from introduction and did not provide a Cmax until approximately 60-minutes from introduction.

The modified polar continuous phase of the MOIW microemulsion is believed to allow the modified oil phase droplets of the MOIW microemulsion to incorporate and retain a high alcohol content. Thus, the modified polar continuous phase of the MOIW microemulsion is believed to force the alcohol into the oil and oil-soluble species residing within the interior of the monolayer walls formed from a phospholipid and a polyethylene glycol derivative, thus into the hydrophobic core of the modified oil droplets, while the modified polar continuous phase including the sugar or sugar alcohol and water resides external to the monolayer.

Unlike the water continuous phase of a conventional OIW emulsion, the sugar or sugar alcohol of the modified polar continuous phase of the MOIW microemulsion does not readily form an azeotrope with alcohol, and thus has a reduced ability to extract the alcohol from the oil droplets in relation to water. The hydrophobic portion of the monolayer wall formed from the tails of the phospholipid in combination with the polyethylene glycol derivative in the described ratios also are believed to reduce alcohol loss from the oil droplets in relation to conventional OIW emulsions.

The retained high alcohol content of the modified oil phase droplets provided by the combination of the modified polar continuous phase with the hydrophobic monolayer is believed to increase the solubility of the oil-soluble species in the modified oil droplets of the MOIW microemulsion in relation to conventional OIW emulsions. This enhanced solubility of the oil-soluble species in the modified oil droplets of the MOIW is believed to reduce dissociation (e.g. recrystallization, precipitation, and like—thus separation) of the oil-soluble species from the oil droplets of the MOIW microemulsion during storage, thus making the MOIW microemulsion a shelf-stable microemulsion that preferably is visually clear and more preferably transparent. Additionally, the enhanced solubility of the oil-soluble species in the modified oil droplets of the MOIW is believed to deliver a greater amount of the oil-soluble species to the bloodstream per unit volume of the MOIW microemulsion in relation to conventional OIW emulsions.

In the MOIW microemulsion, modified oil phase droplets including the oil-soluble species have an average droplet diameter of 1 to 100 nanometers and a preferable average droplet diameter of 5 to 50 nanometers. More preferably, the modified oil phase droplets of the MOIW microemulsion have an average droplet diameter of 10 to 30 nanometers.

The oil-soluble species of the MOIW microemulsions maybe delivered trans-mucosal (e.g. oral) via the MOIW microemulsion. Preferably, the MOIW microemulsion including the oil-soluble species is ingestible and edible.

The MOIW microemulsion preferably includes a ratio of phospholipid, to oil, to polyethylene glycol derivative, to alcohol, to sugar or sugar alcohol, and to water of 1:2:0.6-3.3:4:7-9:2-3.5 by weight, with deviations up to 20% by weight being included, and with deviations up to 10% by weight being more preferred, thus 1:2:0.6-3.3:4:7-9:2-3.5±20% by weight or 1:2:0.6-3.3:4:7-9:2-3.5±10% preferred by weight.

The oil-soluble species is preferably included in the MOIW microemulsion at a ratio of oil to oil-soluble species of 1:0.05 to 1:0.4 by weight, with a ratio of oil to oil-soluble species of 1:0.1 to 1:0.3 by weight being preferred with deviations up to 10% by weight being included, and with deviations up to 5% by weight being more preferred, thus 1:0.05 to 1:0.3±10% by weight or 1:0.05 to 1:0.3±5% preferred by weight.

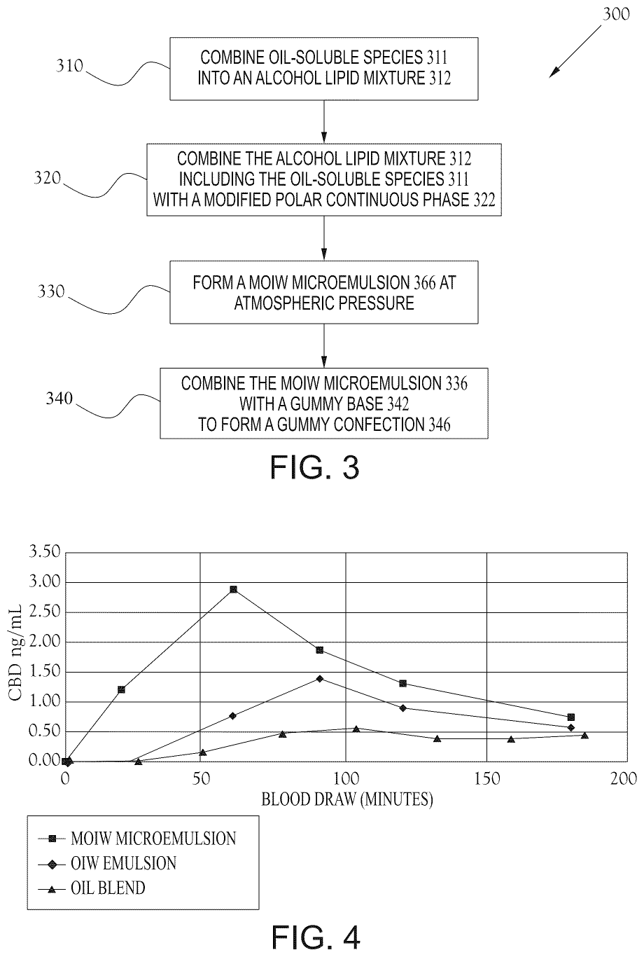

FIG. 3 represents a method 300 of making a MOIW microemulsion 336 incorporated into a gummy confection, where the MOIW microemulsion 336 includes an oil-soluble species 311. In addition to the oil-soluble species 311, the MOIW microemulsion 336 may include additional deliverables that are soluble in water or oil. The MOIW microemulsion 336 is visually clear and preferably transparent.

In 310, the oil-soluble species 311 is combined into an alcohol-lipid mixture 312 including a polyethylene glycol derivative, a phospholipid, an oil, and an alcohol. In 320, the alcohol-lipid mixture 312 including the oil-soluble species 311 is combined with a modified polar continuous phase 322 including the sugar or sugar alcohol and water. The alcohol-lipid mixture 312 including the oil-soluble species 311 maybe considered a modified oil phase dispersed in the modified polar continuous phase 322, which may be thought of as a modified water phase.

In 330, the MOIW microemulsion 336 including the oil-soluble species 311 is formed by mixing at atmospheric pressure. Unlike in nanoemulsions, the MOIW microemulsion 336 may be formed at atmospheric pressure without needing the energy of elevated pressures and/or shear forces to form. Although the MOIW microemulsion 336 could be formed using high energy forces, such as elevated pressure and/or shear forces as used in forming nanoemulsions, the result eventually will be the MOIW microemulsion 336, as unlike in a nanoemulsion that begins the dissociation process after formation—even if dissociation is very slow, the MOIW microemulsion 336 is thermodynamically stable at room temperature and pressure after formation. Thus, formation of the MOIW microemulsion 336 dispenses with the undesirable use of high energy forces during formation, and is shelf-stable after formation.

While the method 300 represents the oil-soluble species 311 first being combined with the alcohol-lipid mixture 312, the alcohol-lipid mixture 312 and the polar continuous phase 322 may first be combined and the oil-soluble species 311 then added to form the MOIW microemulsion 336 (not shown). This step rearrangement is possible as the modified oil and modified polar continuous phases will “self-assemble” droplets including the oil-soluble species to form the MOIW microemulsion 336 at atmospheric pressure.

The oil-soluble species 311 is a liquid at room temperature and pressure, however at high purities, such as above 55% purity by weight, the oil-soluble species 311 may be or may include a crystalline solid. Once solubilized in oil, the oil-soluble species 311 will remain solubilized in the oil at room temperature and pressure. The oil-soluble species 311 preferably includes Cannabis extracts and/or terpenes.

The oil-soluble species 311 is solubilized in the droplets of the MOIW microemulsion 336, thus in the alcohol lipid mixture 312. The alcohol-lipid mixture 312 is preferably configured so that the oil-soluble species 311 is more soluble in the alcohol-lipid mixture 312 than in the oil alone of the MOIW microemulsion 336.

Preferably, the oil-soluble species 311 constitutes from 1% to 6% of the MOIW microemulsion 336 by weight. When the oil-soluble species 311 is substantially Cannabis extracts, to provide a visually clear MOIW microemulsion with the widest range of oil-soluble species, weight percentages of the oil-soluble species 311 from 1% to 4% are preferred, with weight percentages from 1% to 3% being more preferred. When the oil-soluble species 311 is substantially terpenes, higher weight percentages within the 1% to 6% range may be used in the microemulsion 336 than when the oil-soluble species is substantially Cannabis extract and maintain a visually clear MOIW microemulsion. When the oil-soluble species 311 is substantially one or more oil-soluble vitamins, weight percentages within the 0.01% to 6% range may be used in the microemulsion 336 to form a visually clear MOIW microemulsion.

These stated weight percentages for the oil-soluble species 311 are in the context of the oil-soluble species 311 solubilized in the droplets of the MOIW microemulsion 336, not suspended in the emulsion liquid or otherwise dissociated from the droplets. An example of this situation is discussed in the context of Example 3 below, where a commercially purchased product either had an incorrect oil-soluble species content on the label, or approximately 70% of the oil-soluble species in the product had dissociated from the emulsion.

Cannabis extracts are oily extracts from a plant of the Cannabis genus. Preferable Cannabis extracts include cannabidiol (CBD), tetrahydrocannabinol (THC), and other cannabinoids including cannabinol (CBN), cannabigerol (CBG), tetrahydrocannabivarin (THCV), cannabidivarin (CBDV), and cannabichromene (CBC). Preferred Cannabis extracts include at least 30% by weight CBD and/or THC, while more preferred Cannabis extracts include at least 60% by weight CBD and/or THC. Most preferred Cannabis extracts include at least 80% by weight CBD and/or THC.

Preferable terpenes include monoterpenes (incorporate two isoprene units and have the molecular formula C₁₀H₁₆), monoterpenoids, diterpenes (incorporate four isoprene units and often have the molecular formula C₂₀H₃₂), and diterpenoids. Preferable terpenes for inclusion in the microemulsion 336 include limonene, pinene, linalool, beta-caryophyllene, retinol, phytol, myrcene, humulene, ocimene, terpinolene, geraniol, and geranylgeraniol.

Preferable oil-soluble vitamins include Vitamin A, Vitamin D, Vitamin E, Vitamins K1, and Vitamin K2. Oil-soluble carotenoids, such as lutein, zeaxanthin, lycopene, and beta carotene also may be included.

The alcohol lipid mixture 312 optionally may include an alcohol-soluble deliverable that is a solid at room temperature and pressure. Thus, unlike the oil-soluble species 311 that is a liquid at room temperature and pressure or heated and solubilized in oil as previously described, the alcohol-soluble deliverable is a solid at room temperature and pressure. Preferably, the alcohol-soluble deliverable is less soluble in the oil than the oil-soluble species 311. Such alcohol-soluble deliverables are solubilized in the modified oil phase droplets of the microemulsion, thus in the alcohol lipid mixture 312 with the oil-soluble species 311.

Alcohol-soluble deliverables include some plant sterols, some polyphenols, and some anti-microbials. Preferable plant sterols include Tribulus terrestris and yohimbe. Preferable polyphenols include resveratrol, pterostilbene, curcumin, boswellia, and quercetin. Preferable anti-microbials include artemisinin, monolaurin, and andrographis. Preferably, these alcohol-soluble deliverables are incorporated into the alcohol lipid mixture 312 of the MOIW microemulsion 336 as a solid in powder form.

The modified polar continuous phase 322 may include a water-soluble deliverable specie or species that is more soluble in water than the oil-soluble species 311. Such water-soluble deliverables are solubilized in the modified polar continuous phase 322 of the MOIW microemulsion 336. Thus, in the carrier liquid of the MOIW microemulsion 336.

Water-soluble deliverable specie or species include Vitamin C (ascorbic acid and/or sodium ascorbate), the B Vitamins, trimethylglycine, gamma-aminobutyric acid (GABA), theanine, elderberry, and zinc citrate. Skull cap is a plant extract believed to have anti-inflammatory activity that is alcohol-soluble as previously discussed, and can thus be included in the alcohol lipid mixture 312 of the MOIW microemulsion 336, but also has sufficient water-solubility to be a water-soluble deliverable.

The phospholipid and the polyethylene glycol derivative in combination form the boundary between the modified polar continuous phase and the interior of the modified oil phase droplets of the microemulsion 336. To maintain the desired alcohol concentration within the droplets, thus reducing the likelihood of losing the alcohol to the modified polar continuous phase and the associated dissociation of the oil-soluble species from the droplets, the phospholipid, polyethylene glycol derivative, and the ratio between the two are important, as previously discussed.

The phospholipid of the alcohol-lipid mixture 312 is a glycerophospholipid preferably isolated from lecithin. As the phospholipid is preferably a lecithin isolate, the named isolates preferably include 80% (w/w) of the specified phospholipid with the remaining constituents being one or more additional phospholipids isolated from the lecithin or other lecithin isolates.

Preferred phospholipid lecithin isolates include phosphatidylcholine (PC), phosphatidylethanolamine (PE), phosphatidylinositol (PI), ceramide phosphoryl ethanolamine (Cer-PE), ceramide phosphoryl choline (SPH), and combinations thereof, with PC, PE, and combinations thereof being more preferred. However, all phospholipid lecithin isolates are unexpectedly not interchangeable in forming shelf-stable and visually clear MOIW microemulsions, as the phosphatidylserine (PS) and phosphatic acid (PA) isolates are not useful when both shelf-stable and visually clear MOIW microemulsions are desired. When the oil-soluble species 311 is Cannabis extracts, the phospholipid is preferably PC.

The phospholipid may be present in the MOIW microemulsion 336 from 2% to 10% on a weight basis. Preferably, the phospholipid constitutes from 4% to 10% of the MOIW microemulsion 336 on a weight basis. When the oil-soluble species is Cannabis extracts, the phospholipid constitutes from 4% to 8% of the MOIW microemulsion 336 on a weight basis.

The polyethylene glycol derivative of the alcohol-lipid mixture 312 may be a polyethylene glycol modified vitamin E, such as tocopheryl polyethylene glycol succinate 1000 (TPGS), polysorbate 40, polysorbate 60, or polysorbate 80. Preferably, the polyethylene glycol derivate is TPGS, polysorbate 60, or polysorbate 80. More preferably, the polyethylene glycol derivative is TPGS or polysorbate 80. When the oil-soluble species is Cannabis extracts, the preferred polyethylene glycol derivative is TPGS.

The polyethylene glycol derivative may be present in the MOIW microemulsion 336 from 5% to 15% on a weight basis. Preferably, the polyethylene glycol derivative constitutes from 6% to 12% of the MOIW microemulsion 336 on a weight basis. When the oil-soluble species is Cannabis extracts, the polyethylene glycol derivative constitutes from 9% to 11% of the MOIW microemulsion 336 on a weight basis.

TPGS, polysorbate 20, polysorbate 40, polysorbate 60, and polysorbate 80 are often thought of as interchangeable surfactants. This was determined not to be the case in the formation of the described MOIW microemulsion 336 when a shelf-stable and visually clear microemulsion is desired.

When used in conjunction with the phospholipid, TPGS resulted in shelf-stable and visually clear MOIW microemulsions at phospholipid to TPGS ratios of approximately 1:0.4 to 1:4 by weight, with preferred shelf-stable MOIW microemulsions being formed at ratios of 1:1.6 to 1:4 by weight. When used in conjunction with the phospholipid, polysorbate 20 did not form shelf-stable and visually clear MOIW microemulsions. When used in combination with the phospholipid, polysorbate 40 resulted in shelf-stable and visually clear MOIW microemulsions at PC to polysorbate 40 ratios of approximately 1:2 to 1:3 by weight, with preferred shelf-stable MOIW microemulsions being formed at a ratio of approximately 1:3 by weight. When used in combination with the phospholipid, polysorbate 60 resulted in shelf-stable and visually clear MOIW microemulsions at phospholipid to polysorbate 60 ratios of approximately 1:2 to 1:4 by weight, with preferred shelf-stable MOIW microemulsions being formed at a ratio of 1:2 to 1:3 by weight. When used in combination with the phospholipid, polysorbate 80 resulted in shelf-stable and visually clear MOIW microemulsions at phospholipid to polysorbate 80 ratios of approximately 1:0.4 to 1:4 by weight, with preferred shelf-stable MOIW microemulsions being formed at a ratio of 1:0.6 to 1:4 by weight.

These results establish that the multiple polyethylene glycol derivatives are unexpectedly not interchangeable in forming shelf-stable and visually clear MOIW microemulsions. In fact, polysorbate 20 is not useful. Furthermore, TPGS and polysorbate 80 are the preferred polyethylene glycol derivatives as in combination with the phospholipid, they provide the desired shelf-stable and visually clear MOIW microemulsions over the widest oil-soluble species concentration range.

The alcohol-lipid mixture 312 preferably includes at least one oil held within the phospholipid/polyethylene glycol derivative monolayer. The oil may be an MCT oil, a citrus oil, and combinations thereof. Preferable MCT oils include caproic acid (hexanoic acid), caprylic acid (octanoic acid), capric acid (decanoic acid), lauric acid (dodecanoic acid), and combinations thereof. More preferred MCT oils include caprylic acid, capric acid, and combinations thereof. Preferred citrus oils include orange oil, lemon oil, and combinations thereof. When the oil-soluble species is Cannabis extracts, the oil is preferably a combination of caprylic and capric acids.

The oil may be present in the MOIW microemulsion 336 from 5% to 15% on a weight basis. Preferably, the oil constitutes from 7% to 13% of the MOIW microemulsion 336 on a weight basis. When the oil-soluble species is Cannabis extracts, the oil constitutes from 9% to 11% of the MOIW microemulsion 336 on a weight basis.

The MOIW microemulsion 336 includes at least one alcohol. The preferable alcohol is food grade as the MOIW microemulsion 336 is preferably edible. Preferably, the alcohol is ethanol, with USP food grade 190 proof (95% ethanol, 5% water) ethanol being more preferred. Alcohol water contents greater than 10% are less preferred, as then the additional water should be considered in relation to the total water content of the MOIW microemulsion 336 to prevent dissociation of the oil-soluble species from the modified oil phase droplets as discussed further below.

The alcohol may be present in the MOIW microemulsion 336 from 5% to 25% on a weight basis. Preferably, the alcohol constitutes from 10% to 23% of the MOIW microemulsion 336 on a weight basis. When the oil-soluble species is Cannabis extracts, the alcohol constitutes from 16% to 22% of the MOIW microemulsion 336 on a weight basis.

The modified oil phase droplets of the MOIW microemulsion 336 may be considered to have a high alcohol content, thus having an oil to alcohol weight ratio from 1:1.5 to 1:4, preferably from 1:1.5 to 1:3 by weight.

The modified polar continuous phase 322 includes a sugar or sugar alcohol and water. By “sugar or sugar alcohol” it is meant a sugar or a sugar alcohol preferably including from 3 to 12 carbon atoms that is a liquid at room temperature or soluble in water at room temperature. Preferable sugars include sucrose, cane sugar, and pure maple syrup, with pure maple syrup being preferred due to the inclusion of tree resins. Preferable sugar alcohols have from 3 to 6 carbon atoms and include glycerol (glycerin).

While one could expect additional sugar alcohols, including xylitol, erythritol, mannitol, and sorbitol to be useful in forming the MOIW microemulsion 336, all sugar alcohols are unexpectedly not interchangeable in forming shelf-stable and visually clear MOIW microemulsions, as xylitol, erythritol, mannitol, and sorbitol are not useful when both shelf-stable and visually clear MOIW microemulsions are desired. Thus, preferred sugar or sugar alcohols include sucrose, cane sugar, pure maple syrup, glycerol, and combinations thereof. More preferred sugar or sugar alcohols include pure maple syrup, glycerol, and combinations thereof. Presently, the most preferred sugar or sugar alcohol is glycerol.

When the sugar or sugar alcohol is glycerol, the ratio of glycerol to water is from 2:1 to 4.5:1 by weight, preferably from 3:1 to 4.5:1 by weight. When the sugar or sugar alcohol is pure maple syrup, sucrose, or cane sugar, and water is present in the syrup or used to solubilize the sucrose or cane sugar, this additional water becomes part of the water constituent of the MOIW microemulsion 336 and is thus included in the sugar or sugar alcohol to water weight ratio as water.

When the sugar or sugar alcohol is glycerol and the total water content of the MOIW microemulsion 336 is >10% to 25%, the glycerol may be present in the MOIW microemulsion 336 from 30% to 55%, preferably from 30% to 50% on a weight basis. When the total water content of the MOIW microemulsion 336 is >10% to 20%, the glycerol may be present in the microemulsion 336 from 30% to 50%, preferably from 30% to 45% on a weight basis. When the oil-soluble species is Cannabis extracts, the glycerol constitutes from 35% to 45% of the MOIW microemulsion 336 on a weight basis.

The water of the polar continuous phase 332 is present in the MOIW microemulsion 336 from >10% to 25% on a weight basis. Preferably, water is present from >10% to 20% on a weight basis in the MOIW microemulsions 336. More preferably, water may be present in the MOIW microemulsion 336 from 11% to 16% on a weight basis. When the oil-soluble species is Cannabis extracts, water is present in the MOIW microemulsion 336 from 12% to 15% on a weight basis. Water contents of 10% and less in the MOIW microemulsion 336 on a weight basis may result in dissociation of the oil-soluble species from the droplets, and thus a non-shelf-stable MOIW microemulsion.

The MOIW microemulsion 336 may optionally include other ingredients or “adjuvants” that are chemically compatible with the oil-soluble species and do not substantially interfere with the separation between the modified oil and water phases of the MOIW microemulsion. Such adjuvants may include thickeners, flavorings, preservatives, antioxidants, electrolytes, perfumes, fillers, and pigments. Other adjuvants maybe used in the MOIW microemulsion 336.

In 340, the MOIW microemulsion 336 is incorporated into a gummy confection 346 by combining the MOIW microemulsion 336 with a gummy base 342. The gummy base 342 is formed by combining solid sugar, liquid syrup, gelling agent, and citric acid with water. The gelling agent may be a hydrophilic or lipophilic gelling agent. Preferably, the gelling agent is hydrophilic. After adding the MOIW microemulsion 336, any desired water-soluble deliverables, and any desired adjuvants to the gummy base 342, the combination is allowed to solidify in a mold. The resulting gummy confection 346 can be removed from the mold with the assistance of a finishing oil and optionally coated with the finishing oil.

The MOIW microemulsion 336 preferably constitutes from 2% to 15% of the gummy confection 346 by weight, more preferably from 5% to 13% by weight, and most preferably from 7% to 11% by weight. The gummy base 342, any optional water-soluble deliverables, and any optional adjuvants make up the remainder of the gummy confection 346.

The solid sugar is preferably cane sugar, but can be other forms of solid sucrose, such as erythritol, xylitol, coconut palm sugar, and tagatose. However synthetic sweeteners, such as STEVIA™, are not useful as the solid sugar. The solid sugar constitutes from 38% to 42%, preferably from 39% to 41%, of the gummy confection 346 by weight.

The liquid syrup is preferably a glucose-based tapioca syrup from cassava root, but can be other forms of liquid dextrose- or glucose-based syrups, such as potato, corn, rice, and wheat based tapioca syrup. Non-glucose-based tapiocas, such as honey (30% glucose) and maple syrup (<10% glucose) are not useful in this regard. The liquid syrup constitutes from 39% to 43%, preferably from 40% to 41%, of the gummy confection 346 by weight.

The gelling agent is preferably gelatin and/or pectin but can be other forms of gelling agent. Gelatin is formed from proteins removed from animal collagen, while pectin is formed from plants, such as from citrus, apple, and sugar beet peels. Preferably, pectin is used to form the gummy confection 346 as it unexpectedly provides superior blood uptake as will be discussed further below. Pectin also has the advantage of not being thermodynamically reversible after solidifying, thus pectin will not reliquefy if heated during storage and/or transport. The gelling agent constitutes from 1% to 4%, preferably from 2% to 4%, of the gummy confection 346 by weight.

While the gummy confection 346 preferably uses citric acid as a pH modifier, other edible acids may be used that are compatible with the structure of the microemulsion 336 and of the gummy confection 346. Other potentially useful edible acids include malic acid, fumaric, acid, and tartaric acid. When citric acid is the pH modifier, the edible acid constitutes from 0.5% to 4%, preferably from 1% to 2%, of the gummy confection 346 by weight.

The adjuvants can be used to alter the taste and color of the gummy confection 346. For example, the gummy confection 346 incorporating the MOIW microemulsion 336 may be modified to include honey and hibiscus flavorings and a bitterness masker to improve the taste of the gummy confection 346. Other or additional flavorings may be used to improve the taste of the gummy confection 346. The gummy confection 346 incorporating the MOIW microemulsion 336 also may be modified to include one or more coloring agents to provide the desired color.

The water included in the gummy confection 346 arising from the gummy base 342 is from 9% to 18%, preferably from 9% to 14% of the gummy confection 346 by weight. The amount of water arising from the MOIW microemulsion 336 is from 0.2% to 6%, preferably from 1% to 4% by weight. Thus, the total weight percent water in the gummy confection is from 9.2% to 22%. The ratio of water arising from the gummy base 342 in relation to the amount of water arising from the MOIW microemulsion 336 incorporated into the gummy confection 346 is relevant to maintain the gummy nature of the confection and to provide the superior bloodstream delivery of the oil-soluble species provided by the MOIW microemulsion in relation to conventional gummy confections.

Too much additional water originating from the gummy base, thus “gummy base water”, will result in a confection that is poorly formed and/or lacks the desired gummy mouthfeel of elasticity and memory. Preferred weight ratios of the microemulsion 336 incorporated into the gummy confection 346 to the gummy base water are from 1:2 to 1:5±10%. More preferred weight ratios of the MOIW microemulsion 336 incorporated into the gummy confection 346 to the gummy base water are from 1:2 to 1:5±5%. Greater amounts of gummy base water result in a loss of structure and mouthfeel for the gummy confection 346.

Additional water up to approximately 20% by weight of the gummy base 342 may be used to assist in including any desired water-soluble deliverables into the gummy base 342 during formation of the gummy base 342. However, the additional water is substantially removed prior to allowing the combination to solidify in the mold to the extent it would exceed the 9% to 18% by weight of the gummy confection 346 that may be attributed to the gummy base water.

Incorporating the oil-soluble deliverables into the gummy confection 346 via the MOIW microemulsion 336, as opposed to directly adding the oil-soluble deliverables to the gummy base 342, as is done with the water-soluble deliverables, is a significant differentiating factor in relation to conventional gummy confections including both water- and oil-soluble deliverables. It is believed that by incorporating the MOIW microemulsion 336 into the gummy confection 346, the enhanced ability of the MOIW microemulsion 336 to deliver oil-soluble species to be bloodstream is retained and is thus enhanced in relation to conventional gummy confections lacking the MOIW microemulsion 336.

In conventional gummy confections, oil-soluble deliverables are often combined with the gelling agents and water-soluble deliverables during formation and may or may not be solubilized. Generally, the oil-soluble deliverables are not solubilized in the constituents of conventional gummy confections, instead being physically “trapped” in the solid matrix that forms upon cooling to form the solid. Such “trapping” often results in non-homogeneity and at a minimum poor distribution of the oil-soluble deliverables throughout the formed solid matrix, often with the oil-soluble deliverables separating and coating the surface of the gummy confection during cooling and/or leaching out of the gummy confection during storage. Such inconsistent distribution of the oil-soluble deliverables throughout the solid matrix of conventional gummy confections is believed a key factor in their inconsistent and reduced bloodstream delivery performance in relation to the described gummy confections.

Such conventional gummy confections are very different from the described MOIW microemulsion including gummy confections as for the gummy confection 346 the oil-soluble deliverables are incorporated into the gummy base 342 via the MOIW microemulsion 336, as opposed to being directly added to the gummy base 342, as done in conventional gummy confections. Thus, for the gummy confection 346, the optional water-soluble but not the oil-soluble deliverables are directly added to the gummy base 342.

The following examples are provided to illustrate one or more preferred embodiments of the invention. Numerous variations can be made to the following examples that lie within the scope of the invention.

### EXAMPLES

### Example 1: Constituents of a MOIW Microemulsion Including Cannabis Extracts as the Oil-Soluble Species

A MOIW microemulsion was prepared having a 1 mL total volume. The MOIW microemulsion included approximately 10 mg of Cannabis extracts including approximately 80% by weight CBD. The MOIW microemulsion also included from 30 mg to 100 mg of PC, from 150 mg to 250 mg of ethanol, from 300 mg to 550 mg of glycerin, and from 50 mg to 150 mg of medium chain triglycerides. TPGS was included to provide the desired physical structures in the MOIW microemulsion. In addition to these ingredients, the MOIW microemulsion included enough water to provide a total emulsion volume of 1 mL.

### Example 2: A Method of Making a MOIW Microemulsion Including Cannabis Extracts

Approximately 10 mg of CBD from Cannabis extracts including approximately 80% by weight CBD was combined in MCT oil and then combined with TPGS, PC, glycerin, and ethanol in water. The combination was then mixed to form a MOIW microemulsion including the Cannabis extracts.

### Example 3: Comparative Blood Uptake Rates for Intra-Oral Delivery of the Cannabis Extract CBD

Three Cannabis extract liquid carrier systems were compared from a CBD blood uptake rate perspective. While CBD was used in this example as the oil-soluble species due to availability, the blood uptake rate data is believed to be similar for other oil-soluble species, such as THC, other cannabinoids, and terpenes. The first carrier system was a MOIW microemulsion consistent with Examples 1 and 2. The second carrier system was a conventional oil blend. The third carrier system was a conventional OIW emulsion.

The oil blend second carrier system was prepared by combining approximately 1.5% by weight Cannabis extract including approximately 80% by weight CBD with approximately 98.5% by weight hemp seed oil to provide a conventional oil-only product. While hemp seed oil was used for this example, other oils including sunflower, olive, and MCT are expected to provide similar blood uptake rates.

The conventional OIW emulsion third carrier system was a commercially available product and according to the label included approximately 17 mg/mL of the Cannabis extract CBD with the emulsion constituents being water, glycerin, MCT oil, natural lipids, the polysaccharides Xanthan and Acacia gum, Stevia, and potassium sorbate. The percentages of the OIW emulsion constituents were not known as such information was not provided on the product label; however, the product was known to be a conventional, non-visually clear OIW emulsion. Independent testing revealed that this commercially obtained OIW emulsion product included 4.8 mg/mL CBD in the liquid, as opposed to the claimed approximately 17 mg/mL.

On an empty stomach, human subjects placed 1.2 mL of the MOIW microemulsion, 1 mL of the conventional oil blend, or 2.14 mL of the OIW emulsion including the Cannabis extract CBD under the tongue. Slightly different volumes were used for each of the three carrier systems, so all dosages included approximately 10 mg of CBD. For the commercially obtained OIW emulsion, the 2.14 mL was used to provide the desired 10 mg CBD dose in view of the actual 4.8 mg/mL liquid concentration. The subject held each liquid under the tongue for approximately 30 seconds to 2 minutes before swallowing.

Blood samples were collected before the carrier system liquid was administered and at varying time intervals between approximately 20 and 180 minutes after oral administration of the carrier system liquid. The collected blood samples were analyzed for the concentration of CBD using LCMS.

FIG. 4 provides the results of a CBD blood uptake rate and concentration analysis in graphical form for the liquid carrier systems. The time after administration of the carrier system liquid when the blood sample was collected is represented on the X-axis, while the average nanograms (ng) of CBD per milliliter (mL) determined for the blood samples is represented on the Y-axis.

The MOIW Microemulsion line represents the blood uptake concentrations obtained with a MOIW microemulsion in accord with Examples 1 and 2. The oil blend line represents the blood concentrations obtained for the oil blend. The OIW Emulsion line represents the blood concentrations obtained for the conventional OIW emulsion.

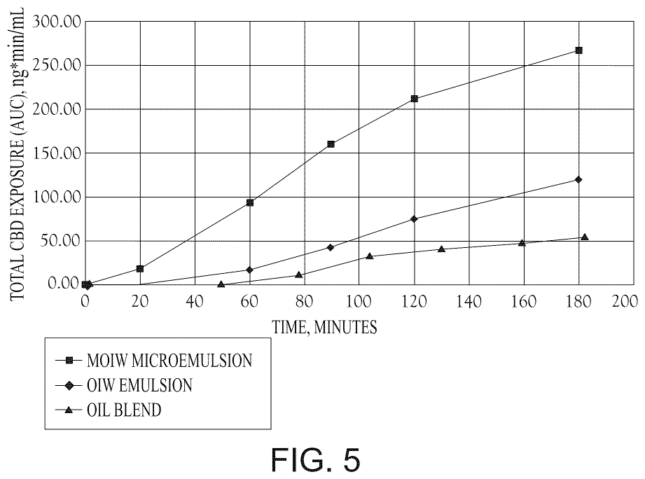

FIG. 5 provides the cumulative Area Under the Curve (AUC) values in ug*(min/mL) determined from a bloodstream concentration analysis in graphical form for the liquid carrier systems. The AUC values provide a measure of the cumulative amount of CBD in the bloodstream, thus total exposure across a period of time.

The superiority of the MOIW microemulsion at rapidly delivering CBD to the bloodstream in relation to the oil blend is readily apparent from a blood uptake perspective. Relative CBD bioavailability between the different delivery systems was determined at different times after intra-oral introduction by multiplying the AUC up to the selected time point by the amount of CBD in the selected Cannabis extract carrier system and dividing by a control value likewise calculated, thus (AUC1*carrier system1)/(AUCcontrol*carrier system control) at the selected time—thus calculating AUC as a cumulative amount of CBD in the bloodstream. As this calculation divides the total amount of CBD delivered by the numerator carrier system liquid by the total amount of CBD delivered by the denominator carrier system liquid, the resulting values relate to how many times more CBD was delivered by the numerator carrier system liquid in relation to the denominator carrier system.

The results from the AUC calculations are provided below in Table 1 for the MOIW microemulsion as the numerator and the oil blend as the denominator with estimation due to the slight variability in the withdrawal time of the blood samples.

TABLE 1

| Time | MOIW/Oil-Only |
|---|---|
| 0 | N/A |
| 20 | — |
| 60 | 22 |
| 90 | 10 |
| 120 | 7 |
| 180 | 5 |

At the 20-minutes after oral introduction, a valid delivery comparison value could not be determined as the CBD measured in the blood resulting from the oil blend introduction was below the sensitivity of the LCMS instrument. As the MOIW microemulsion achieved an approximately 1.2 ng/mL blood concentration after introduction, and the oil blend achieved an essentially 0 blood concentration, the ability of the MOIW microemulsion to provide a significantly enhanced blood uptake rate in relation to the oil blend was established at the 20-minute time. Thus, with oral introduction of a 1 mL dose of the MOIW microemulsion, the MOIW microemulsion can provide within 20-minutes a CBD concentration from 0.3 to 1.5 ng/mL, preferably from 0.6 to 1.5 ng/mL, and more preferably from 0.8 to 1.5 ng/mL.

The significance of the MOIW microemulsion's 20-minute rapid delivery is seen at the 60-minute time point where the MOIW microemulsion had cumulatively delivered approximately 22 times more CBD to the bloodstream than the oil blend (peak concentrations of 2.9 ng/mL for the MOIW microemulsion vs. approximately 0.16 ng/mL for the oil blend). In fact, it was not until the 90-minute time point that the rate of increasing cumulative delivery provided by the MOIW microemulsion in relation to the oil blend was observed to slow. By cumulatively provided it is meant that the total bloodstream available CBD from introduction of the liquid carrier system until the selected time. It was not until the approximately 180-minute mark that the oil blend delivered CBD to the bloodstream at a rate comparable to that of the MOIW microemulsion.

As for the oil blend, the superiority of the MOIW microemulsion at rapidly delivering CBD to the bloodstream in relation to the conventional OIW emulsion also is apparent from a blood uptake perspective. The results from the AUC calculations are provided below in Table 2 for the MOIW microemulsion as the numerator and the OIW emulsion as the denominator with estimation due to the slight variability in the withdrawal time of the blood samples.

TABLE 2

| | MOIW/OIW |
|---|---|
| Time | Emulsion |
| 0 | N/A |
| 20 | |
| 60 | 6.7 |
| 90 | 3.6 |
| 120 | 2.7 |
| 180 | 2.2 |

At the 20-minutes after oral introduction, a valid delivery comparison value could not be determined as the CBD measured in the blood resulting from the OIW emulsion introduction was below the sensitivity of the LCMS instrument. As the MOIW microemulsion achieved an approximately 1.2 ng/mL blood concentration after introduction, and the OIW emulsion achieved an essentially o blood concentration, the ability of the MOIW microemulsion to provide a significantly enhanced blood uptake rate in relation to the OIW emulsion was established at the 20-minute time.

The significance of the MOIW microemulsion's 20-minute rapid delivery is readily seen at the 60-minute time point where the MOIW microemulsion had cumulatively delivered almost 7 times more CBD to the bloodstream than the OIW emulsion (peak concentrations of 2.9 ng/mL for the MOIW microemulsion vs. approximately 0.43 ng/mL for the oil blend). As with the oil blend, it was not until the approximately 180-minute mark that the OIW emulsion is delivering CBD to the bloodstream at a rate comparable to that of the MOIW microemulsion.

Of interest is that between the 60- and 90-minute time points the rate of increasing cumulative delivery decreased for the MOIW microemulsion while continuing to approximately linearly increase for the OIW emulsion. This is believed to illustrate the difference between the MOIW microemulsion's enhanced ability to intra-orally deliver to the bloodstream verses the OIW emulsion's favored gastrointestinal bloodstream delivery pathway. The substantially more rapid (20-minute time) blood uptake provided by the MOIW microemulsion in relation to the OIW emulsion shows the MOIW microemulsion's ability to substantially “bypass” the gut. Both the oil blend and the OIW emulsion are believed to substantially deliver the oil-soluble species via the gut while the MOIW microemulsion is believed to substantially intra-orally deliver the oil-soluble species via the mouth and esophagus.

### Example 4: Total Exposure for Intra-Oral Delivery of the Cannabis Extract CBD

The three Cannabis extract oral carrier systems from Example 3 also were compared from a CBD total exposure perspective. With each extract carrier system approximately 10 mg of CBD was introduced. Thus, by comparing the AUC values for each of the three extract carrier systems within a selected timeframe, the total amount of Cannabis extract to which a subject was exposed may be compared.

At the 60-minute time, the AUC was 94 ng*min/mL for the MOIW microemulsion, 14 for the conventional OIW emulsion, and 4.2 for the oil blend. Thus, at the 60-minute time point, of the approximately 10 mg of CBD consumed by the subject, the MOIW microemulsion had exposed the subject to nearly 7 times as much CBD as had the conventional OIW emulsion and approximately 22 times as much CBD as had the oil blend. The MOIW microemulsion was able to deliver from 5 to 9 times, preferably from 6 to 8 times, more CBD to the bloodstream after 60-minutes than the OIW emulsion. The MOIW microemulsion was able to deliver from 18 to 24 times, preferably from 20 to 22 times, more CBD to the bloodstream after 60-minutes than the oil blend.

At the 180-minute time, the AUC was 273 ng*min/mL for the MOIW microemulsion, 122 for the conventional OIW emulsion, and 58 for the oil blend. At the 180-minute mark the MOIW microemulsion and OIW emulsion carrier systems are delivering CBD to the bloodstream at similar rates, however from a cumulative and thus total delivery perspective, the MOIW microemulsion delivered more than twice as much CBD to the bloodstream as the OIW emulsion. At the 180-minute time, the oil blend carrier system is delivering CBD to the bloodstream at a somewhat faster rate than the MOIW microemulsion, however from a cumulative and thus total delivery perspective, the MOIW microemulsion delivered nearly 5 times as much CBD to the bloodstream as the oil blend.

At the 180-minute time, of the approximately 10 mg of CBD consumed by the subject, the MOIW microemulsion had exposed the subject to more than twice as much CBD as had the conventional OIW emulsion and almost 5 times as much CBD as had the oil blend. Thus, over the 180-minute timeframe, the MOIW microemulsion delivered at least 80%, preferably at least 100%, more CBD to the bloodstream as did the OIW emulsion and at least 4 times, preferably at least 5 times, as much CBD to the bloodstream as did the oil blend.

The data shows that for a subject to obtain similar Cannabis extract exposure over a 3-hour timeframe to that provided by approximately 1 mL of the MOIW microemulsion, the subject would have to consume nearly 5 mL of the conventional OIW emulsion, or nearly 5 mL of the oil blend. Even in these “large dose” circumstances, the conventional OIW emulsion and the oil blend would be unlikely to provide the same 20-minute rapid onset bloodstream concentrations provided by the MOIW microemulsion. Thus, the ability of the MOIW microemulsion to deliver the Cannabis extract rapidly and efficiently to the bloodstream at substantially smaller dosing volumes was established.

### Examples: Blood Uptake Consistency for Intra-Oral Delivery of the Cannabis Extract CBD

The MOIW microemulsion and oil blend Cannabis extract carrier systems from Example 3 were compared from a CBD bloodstream uptake consistency perspective for 10 human subjects. Blood samples were collected from the subjects at varying time intervals between approximately 20 and 180 minutes after intra-oral administration of the carrier system liquid. The collected blood samples were analyzed for the concentration of CBD using LCMS. The standard deviation between the CBD blood concentrations was determined at each time point for the collected samples. The calculated standard deviation was then divided by the average CBD blood concentration determined at each time point to provide the percent standard deviation of the CBD blood concentrations. The determined values are provided below in Table 3.

TABLE 3

| Approximate Time | MOIW | |
|---|---|---|
| Minutes | Microemulsion | Oil-Only |
| 50-60 | 68% | 242% |
| 77-90 | 67% | 86% |
| 120-135 | 49% | 126% |
| 155-180 | 77% | 86% |
| AVG | 65% | 135% |

When the percent standard deviations are averaged for the MOIW microemulsion, an average percent standard deviation of 65% was determined. When the percent standard deviations are averaged for the oil blend, an average percent standard deviation of 135% was determined. Thus, the MOIW microemulsion provided less than half the variability in blood uptake in relation to the blood uptake provided by the oil blend. This substantial increase for the dosing reproducibility provided by the MOIW microemulsion in relation to the oil blend is believed attributable to the MOIW microemulsion's ability to provide relatively consistent intra-oral delivery in relation to the oil blend, which provides inconsistent blood uptake from reliance on GI adsorption.

We believe that the low and inconsistent uptake provided by conventional delivery systems coupled with the varying absorption ability of different subjects are factors in the inconsistent efficacy reports obtained from subjects using conventional oral cannabinoid delivery systems.

### Example 6: Method of Making a MOIW Microemulsion Gummy Confection Including Cannabis Extracts as the Oil-Soluble Species

A MOIW microemulsion including Cannabis extracts (CBD oil 98% by weight) was prepared in accord with Example 1 in a volume larger than 1 mL. A first gummy base component was formed by combining approximately 10-15 grams (g) of a first component of solid cane sugar with approximately 2 to 3 g of pectin. The solid cane sugar and pectin were added to hot water and dissolved. A second gummy base component was formed by combining approximately 40 to 45 g of tapioca syrup with 28 to 34 g of a second component of solid cane sugar. The solid cane sugar and tapioca syrup were combined with water and heated to dissolve the solids. The first and the second gummy base components were then combined with stirring and heating continued.

The MOIW microemulsion (7-10 g) and the water-soluble deliverables GABA (approximately 1 g), Theanine (approximately 1 g), and skull cap (approximately 1-2 g) with enough citric acid to provide a pH from 3.3 to 3.5 were then combined with the combined first and second gummy base components. Adjuvants also were added to flavor, color, and reduce the bitterness of the combination.

The combination was heated until the water content of the combination decreased to 15% to 18% by weight. The combination was then transferred to multiple molds and allowed to cool. The resulting gummy confections were removed from the molds with the assistance of an organic finishing oil.

A similar gummy confection was made through omission of the water-soluble deliverables. Similar gummy confections were made where the pectin was replaced with gelatin.

### Example 7: Method of Making a MOIW Microemulsion Gummy Confection Including Oil-Soluble Vitamins as the Oil-Soluble Deliverable and Water-Soluble Vitamins as the Water-Soluble Deliverable

A MOIW microemulsion including oil-soluble vitamins (Vitamins A, D3, E as tocotrienols, K1, and K2—approximately 3 g total) instead of Cannabis extracts was prepared in accord with Example 1 in a volume larger than 1 mL. A first gummy base component was formed by combining approximately 10 to 15 g of solid cane sugar with approximately 2 to 3 g of pectin. The solid cane sugar and pectin were added to hot water and dissolved. A second gummy base component was formed by combining approximately 40 to 45 g of tapioca syrup with 28 to 34 g of solid cane sugar. The solid cane sugar and tapioca syrup were combined with water and heated to dissolve the solids. The first and the second gummy base components were then combined with stirring and boiling continued.

The MOIW microemulsion (6-8 g) and the water-soluble soluble deliverables Vitamin C, B1, B2, B3, B5, B6, B7, B9, and B12, and Trimethylglycine with enough citric acid to provide a pH from 3.3 to 3.5 were then combined with the combined first and second gummy base components. Adjuvants also were added to flavor and color the combination.

The combination was allowed to boil until the water content decreased to the point where the water content provided by the first and the second gummy base components constituted from 9% to 13% by weight of the combination. The combination was then transferred to multiple molds and allowed to cool. The resulting gummy confections were removed from the molds with the assistance of an organic finishing oil.

### Example 8: Method of Making a MOIW Microemulsion Gummy Confection Including Oil-Soluble Vitamins as the Oil-Soluble Deliverable and Water-Soluble Deliverables

A MOIW microemulsion including oil-soluble vitamins (Vitamins A, D3, E as tocotrienols, K1, and K2—approximately 3 g total) instead of Cannabis extracts was prepared in accord with Example 1 in a volume larger than 1 mL. A first gummy base component was formed by combining approximately 10 to 15 g of a first component of solid cane sugar with approximately 2 to 3 g of pectin. The solid cane sugar and pectin were added to hot water and dissolved. A second gummy base component was formed by combining approximately 40 to 45 g of tapioca syrup with 28 to 34 g of a second component of solid cane sugar. The solid cane sugar and tapioca syrup were combined with water and heated to dissolve the solids. The first and the second gummy base components were then combined with stirring and boiling continued.

The MOIW microemulsion (6 to 8 g) and the water-soluble soluble deliverables Vitamin C as ascorbic acid, sodium ascorbate, or a combination thereof, elderberry powder, and zinc citrate with enough citric acid to provide a pH from 3.3 to 3.5 were then combined with the combined first and second gummy base components. Adjuvants also were added to flavor the combination.

The combination was allowed to boil until the water content decreased to the point where the water content provided by the first and the second gummy base components constituted from 9% to 13% by weight of the combination. The combination was then transferred to multiple molds and allowed to cool. The resulting gummy confections were removed from the molds with the assistance of an organic finishing oil.

### Example 9: Comparative Blood Uptake Rates for Gummy Delivery of the Cannabis Extract CBD

A MOIW microemulsion gummy confection in accord with Example 6 and a similar MOIW microemulsion gummy confection where the pectin was substituted with gelatin were formed as Cannabis extract delivery systems. While the Cannabis extract CBD was used in this example as the oil-soluble species for delivery, the blood uptake rate data is believed applicable for other oil-soluble species, such as THC, other cannabinoids, terpenes, and oil-soluble vitamins.

A commercially available pectin-based CBD gummy confection was obtained where the label stated the confection to include 20 mg of CBD per gummy confection with other label constituents including sugar, corn syrup, pectin, MCT oil, and natural flavorings and colors. An oil blend CBD carrier system was prepared as previously discussed in Example 3.

On an empty stomach, human subjects consumed through chewing and ingesting enough of the gummies made in accord with Example 6 so that 12 mg of CBD was consumed, which was approximately one gummy. An amount of the commercially available CBD gummy confection (a portion of a gummy) and the oil blend were similarly consumed to provide the same 12 mg CBD dose. Thus, all subjects consumed the same, approximately 12 mg dose of CBD with the four different delivery systems.

Blood samples were collected before consuming the CBD delivery systems and at varying time intervals between approximately 20- and 240-minutes after consumption. The collected blood samples were analyzed for the concentration of CBD using LCMS.

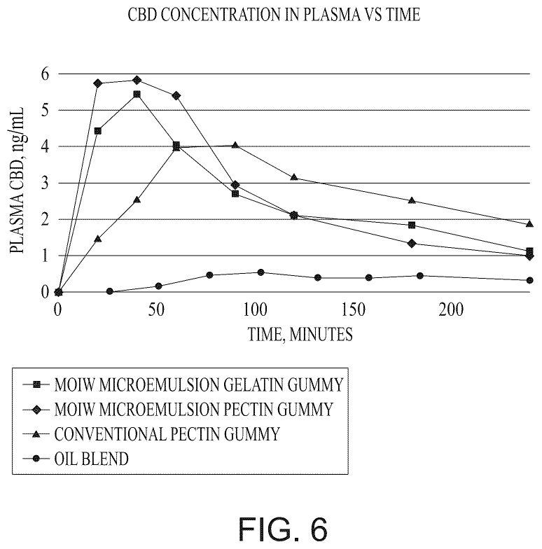

FIG. 6 provides the results of a CBD blood uptake rate and concentration analysis in graphical form for the gummy confections and the oil blend. The time after consumption of the Cannabis extract delivery systems when the blood sample was collected is represented on the X-axis, while the average nanograms (ng) of CBD per milliliter (mL) determined for the collected blood samples is represented on the Y-axis.

The MOIW Microemulsion Gelatin and Pectin Gummy lines represent the blood uptake concentrations obtained from the MOIW microemulsion gummy confections in accord with Example 6. The Conventional Pectin Gummy line represents the blood concentrations obtained for the conventional CBD gummy lacking the MOIW microemulsion. The oil blend line represents the blood concentrations obtained for the oil blend.

The superiority of the gummy confections including the MOIW microemulsion at rapidly delivering CBD to the bloodstream in relation to the conventional gummy confection or the oil blend is readily apparent from a blood uptake perspective, where an over 300% increase in CBD blood concentration was observed at the 20-minute time duration for the MOIW microemulsion gummy confections in comparison to the conventional gummy confection. At the 20-minute time, the gummy confections including the MOIW microemulsion delivered from 4 to 6 ng/mL of the oil-soluble deliverable CBD to the bloodstream, while the conventional gummy confection delivered 1.45 ng/mL. The oil blend delivered unmeasurable CBD to the bloodstream at the 20-minute time duration. Thus, the MOIW microemulsion gummy confection provided an oil-soluble species blood concentration 2 to 4 times greater than that provided by the conventional gummy confection 20-minutes after

### INTRODUCTION

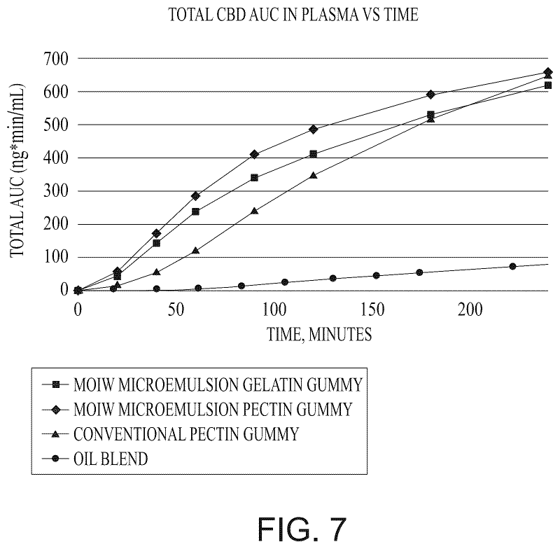

FIG. 7 provides the cumulative Area Under the Curve (AUC) values in (ng*min/mL) determined from a bloodstream concentration analysis in graphical form for the gummy confections and the oil blend. The AUC values provide a measure of the cumulative amount of CBD in the bloodstream, thus total exposure across a time duration.

Relative CBD bioavailability between the different delivery systems was determined at different times after consumption by multiplying the AUC up to the selected time point by the amount of CBD in the selected Cannabis extract delivery system and dividing by a control value likewise calculated, thus (AUC1*carrier system1)/(AUCcontrol*carrier system control) at the selected time—thus calculating AUC as a cumulative amount of CBD in the bloodstream. As this calculation divides the total amount of CBD delivered by the numerator delivery system by the total amount of CBD delivered by the denominator delivery system at a selected time, the resulting values relate to how many times more CBD was delivered by the numerator delivery system in relation to the denominator delivery system at the selected time.

The results from the AUC calculations are provided below in Table 4 for the MOIW microemulsion gummy confections (pectin, gelatin) as the numerator and the oil blend as the denominator with estimation due to the slight variability in the withdrawal time of the blood samples.

TABLE 4

| | MOIW | MOIW |
|---|---|---|
| Time | Pectin/Oil-Blend | Gelatin/Oil-Blend |
| 0 | N/A | N/A |
| 20 | | — |
| 40 | 85.4 | 70.7 |
| 60 | 28.0 | 23.4 |
| 90 | 17.7 | 14.6 |
| 120 | 13.2 | 11.2 |
| 180 | 12.6 | 11.3 |
| 240 | 11.4 | 10.7 |

At 20-minutes after consumption, a valid delivery comparison value could not be determined as the CBD measured in the blood resulting from the oil blend was below the sensitivity of the LCMS instrument. As the MOIW microemulsion gummy confections achieved an approximately 5 ng/mL average blood concentration 20-minutes after consumption, and the oil blend achieved an essentially o blood concentration, the ability of the MOIW microemulsion gummy confections to provide a significantly enhanced blood uptake rate in relation to the oil blend was established at the 20-minute time. Thus, the MOIW microemulsion including gummy confections can provide a CBD blood concentration within 20-minutes of eating the gummy confection of at least 2 to 6 ng/mL, preferably at least 3 to 6 ng/mL, and more preferably at least 4 to 6 ng/mL.

The significance of the MOIW microemulsion gummy confections' 20-minute rapid delivery is seen at the 40-minute time point where the MOIW microemulsion gummy confections had cumulatively delivered an average of approximately 78 times (85.4+70.7/2) more CBD to the bloodstream than the oil blend (peak average concentration of 5.6 ng/mL for the MOIW microemulsion gummy confections vs. approximately 0.16 ng/mL for the oil blend).

The difference in the delivery profiles between the MOIW microemulsion gummy confections and the oil blend also is evidenced by the Cmax values in relation to time. Cmax values are the time during the analysis where the maximum concentration of CBD in the bloodstream was observed. For the MOIW microemulsion pectin-based gummy confection, a Cmax of 5.82 ng/mL was observed at 40-minutes post consumption, while a Cmax of 5.44 ng/mL was also observed at 40-minutes for the MOIW microemulsion gelatin-based gummy confection. Thus, both MOIW microemulsion gummy confections provided a bloodstream Cmax of the oil-soluble deliverable within 30 to 50-minutes post consumption of the confection.

The conventional gummy confection achieved a Cmax of 4.03 ng/mL at 90-minutes. A Cmax of 0.54 ng/mL was observed at approximately 100-minutes for the oil blend. The difference in the delivery profile provided by the inclusion of the MOIW microemulsion in a gummy confection is evidenced by the Cmax values in relation to time. For the MOIW microemulsion pectin-based gummy confection a Cmax of 5.82 ng/mL was observed at 40-minutes post consumption. In contrast, the Cmax of 4.03 ng/mL was observed at approximately 90-minutes for the conventional pectin-based gummy confection. Thus, in addition to the conventional gummy confection providing an approximately 30% lower bloodstream Cmax in relation to the MOIW microemulsion gummy confection, the conventional gummy confection did not achieve Cmax until more than twice the time duration post consumption.

The oil blend provided an approximately order of magnitude lower bloodstream Cmax in relation to the MOIW microemulsion gummy confection (0.54. ng/mL vs. 5.82 ng/mL) approximately 100-minutes after consumption. Thus, the oil blend did not achieve Cmax until more than twice the time duration after consumption in relation to the MOIW microemulsion gummy confections.

The fact that the conventional gummy confection achieved Cmax after 90-minutes, thus close to the 100-minute Cmax of the oil blend, demonstrates the similarity in the delivery profiles of the conventional gummy confection and the oil blend in relation to the much shorter 40-minute Cmax of the MOIW microemulsion gummy confections.

The superiority of the MOIW microemulsion gummy confections at rapidly delivering CBD to the bloodstream in relation to the conventional gummy confection also is apparent from a relative bioavailability perspective. The results from the AUC calculations are provided below in Table 5 for the MOIW microemulsion pectin-based gummy confection as the numerator and the conventional pectin-based gummy confection as the denominator.

TABLE 5

| | MOIW |
|---|---|
| | Pectin/Conventional |
| Time | Pectin |
| 0 | N/A |
| 20 | 3.9 |
| 40 | 3.2 |
| 60 | 2.4 |
| 90 | 1.7 |
| 120 | 1.4 |
| 180 | 1.1 |
| 240 | 1.0 |

At 20-minutes after consumption, the MOIW microemulsion gummy confection had delivered nearly four times as much CBD to the bloodstream as had the conventional gummy confection. As the MOIW microemulsion gummy confection achieved an approximately 5.7 ng/mL blood concentration 20-minutes after introduction, and the conventional gummy confection achieved an approximately 1.4 ng/mL blood concentration, the ability of the MOIW microemulsion gummy confection to provide a significantly enhanced blood uptake rate in relation to the conventional gummy confection was established at the 20-minute time.

The contribution of the MOIW microemulsion to significantly enhance the rate of oil-soluble deliverable uptake for a gummy confection is established. While after a few hours the MOIW microemulsion and conventional gummy confection had delivered approximately the same amount of CBD to the bloodstream, the MOIW microemulsion gummy delivered CBD to the bloodstream approximately four times faster at the 20-minute interval and was still delivering the CBD nearly twice as fast at the 90-minute time interval.

The significance of the MOIW microemulsion gummy confection's 20-minute initial rapid delivery is readily seen at the later 60-minute time where the MOIW microemulsion gummy confection cumulatively delivered more than twice as much CBD to the bloodstream than the conventional gummy confection (AUC of 285 ng*min/mL for the MOIW microemulsion gummy confection vs. 119 ng*min/mL for the conventional gummy confection). The conventional gummy confection could not attain a similar cumulative uptake to the MOIW microemulsion gummy confection until three to four hours after consumption (AUC of 658 ng*min/mL for the MOIW microemulsion including gummy confection vs. 647 ng*min/mL for the conventional gummy confection at 240-minutes).

### Example 10: Total Exposure for Gummy Confection Delivery of the Cannabis Extract CBD

The gummy confection and oil blend Cannabis extract carrier systems from Example 9 also were compared from a CBD total exposure perspective. With each extract carrier system approximately 12 mg of CBD was introduced. Thus, by comparing the AUC values for each of the Cannabis extract carrier systems within a selected timeframe, the total amount of Cannabis extract to which a subject was exposed maybe compared.

At 60-minutes after consumption, the AUC was 285 ng*min/mL for the MOIW microemulsion pectin-based gummy confection, 238 for the MOIW microemulsion gelatin-based gummy confection, 119 for the conventional gummy confection, and 4.2 for the oil blend. Thus, at the 60-minute time point, of the approximately 12 mg of CBD consumed by each subject, the pectin-based MOIW microemulsion gummy confection exposed the subject to more than twice as much CBD as the conventional pectin-based gummy confection and approximately 68 times as much CBD as the oil blend.

The MOIW microemulsion gummy confections were able to deliver from 1.5 to 2.8 times, preferably from 1.8 to 2.4 times, more CBD to the bloodstream after 60-minutes than the conventional gummy confection. The MOIW microemulsion gummy confections were able to deliver from 20 to 70 times, preferably from 40 to 70 times, more CBD to the bloodstream after 60-minutes than the oil blend.

At 90-minutes after consumption, the AUC was 410 ng*min/mL for the MOIW microemulsion pectin-based gummy confection, 339 for the MOIW microemulsion gelatin-based gummy confection, 239 for the conventional gummy confection, and approximately 15 for the oil blend. Thus, at the 90-minute time point, of the approximately 12 mg of CBD consumed by each subject, the pectin-based MOIW microemulsion gummy confection exposed the subject to close to twice as much CBD as the conventional pectin-based gummy confection and approximately 27 times as much CBD as the oil blend.

At 180-minutes after consumption, the AUC was 589 ng*min/mL for the MOIW microemulsion pectin-based gummy confection, 530 for the MOIW microemulsion gelatin-based gummy confection, 516 for the conventional gummy confection, and 58 for the oil blend. Thus, at 180-minutes post consumption, the conventional gummy confection approached the total or cumulative delivery of the MOIW microemulsion gummy confections, while the oil blend continued to remain approximately an order of magnitude lower.

When the analysis was concluded at 240-minutes, the MOIW microemulsion gummy confections had an average AUC value of 638 (659 pectin, 618 gelatin), the conventional gummy confection had an AUC value of 637, while the value for the oil blend was 79, establishing that the MOIW microemulsion gummy confections had delivered more than eight times as much CBD to the bloodstream than the oil blend over the four hours since consumption. The oil blend was not able during the 240-minute time of the analysis to deliver CBD to the bloodstream at a rate or amount comparable to that of the MOIW microemulsion including gummy confections. The MOIW microemulsion gummy confections delivered a similar cumulative amount of CBD to the bloodstream four hours post consumption.

The data shows that for a subject to obtain similar Cannabis extract exposure over a four-hour timeframe to that provided by the MOIW microemulsion gummy confections, the subject would have to consume nearly 8 mL of the oil blend. Even in this “large dose” circumstance, the oil blend would be unlikely to provide the same 20-minute rapid onset bloodstream concentrations provided by the MOIW microemulsion gummy confections. Thus, the ability of the MOIW microemulsion gummy confections to efficiently deliver the Cannabis extract to the bloodstream at substantially smaller dosing volumes in relation to the oil blend was established.

While conventional gummy confections can provide similar total amounts of Cannabis extract to the bloodstream after four hours, the conventional gummy confection provides a Cmax delivery profile more closely aligned with the oil blend and has an approximately 38% lower Cmax (5.58-4.03/4.03*100%) than the MOIW microemulsion gummy confections. Another significant advantage the MOIW microemulsion provides to gummy confections is that substantially more oil-soluble deliverable is provided in the first 90-minutes after consumption when the MOIW microemulsion gummy confection delivery is starting to significantly decrease as most of the delivery is complete—while at the same 90-minute post consumption time the conventional gummy confection is reaching its delivery peak and starting a slow delivery decline.

### Example 11: Comparison of Liquid and Confection Based Delivery Systems

When the FIG. 4 and FIG. 5 liquid delivery system delivery results are compared to the corresponding FIG. 6 and FIG. 7 confection delivery system delivery results, an overall delivery performance improvement is observed for the confection verses liquid delivery systems. While not wishing to be bound by any particular theory, this performance improvement is believed attributable to the longer mouth-residency time of the confection in relation to an orally consumed liquid and the fact that slightly more deliverable was included in the confections.

However, it was unexpected that the significantly superior bloodstream delivery performance of the liquid MOIW microemulsion in relation to the other liquid carrier systems was transferred to the described MOIW microemulsion including gummy confections. Also surprising was that the Cmax for the MOIW microemulsion including gummy confections was observed at approximately 20- to 40-minutes while the Cmax for the MOIW microemulsion intra-oral liquid was closer to 60-minutes. Also surprising was that the MOIW microemulsion confection showed a more rapid delivery decay after reaching its Cmax (especially for the gelatin) in relation to the MOIW microemulsion liquid, while the OIW emulsion liquid and conventional pectin gummy maintained more comparable delivery decay profiles with time after reaching Cmax.

To provide a clear and more consistent understanding of the specification and claims of this application, the following definitions are provided.

Intra-oral delivery means that a substantial portion of the delivery into the bloodstream that occurs upon oral administration of a liquid including the deliverable occurs by transmucosal absorption through the mouth, throat, and esophagus before the liquid reaches the stomach. For droplets to be considered suitable for intra-oral delivery, the average droplet diameter is at most 125 nm.

An oil-soluble species is a species that is insoluble in water and soluble in medium chain triglyceride (MCT) oils at 50 mg/mL and higher, preferably 100 mg/mL and higher. Oil-soluble species are generally soluble in MCT oils at room temperature and are freely or very soluble in MCT oils at temperatures of 70 degrees Celsius and greater. The term “generally soluble in MCT oils at room temperature” is used because some high purity oil-soluble species are sparingly soluble in MCT oils at room temperature, but are freely or very soluble in the MCT oils above 70 degrees Celsius, and once solubilized in the MCT oils at elevated temperature, will remain solubilized at room temperature. Oil-soluble species are preferably pharmacologically active, more preferably are a drug or a supplement, and neither include nor are water. Thus, liquids and solids may exist that technically are soluble in oil, but because they also are soluble in water or not sufficiently soluble in MCT oils are not “oil-soluble species”.

Phosphatidylcholine (PC) molecules are a subset of the larger set of phospholipids and are commonly used to form liposomes in water. When placed in water without other constituents, PC forms liposomes. In the presence of an oil, the application of sufficient shear forces to the PC liposomes in water can produce monolayer structures, including micelles. PC has a head that is water-soluble and a tail that is much less water-soluble in relation to the head. PC is a neutral lipid, but carries an electric dipole moment of about 10 D between the head and the tail, making the molecule itself polar.

Tocopheryl polyethylene glycol succinate 1000 (TPGS) is generally considered a surfactant having a non-polar, oil-soluble “Vitamin E” tail and a polar, water-soluble polyethylene glycol head. TPGS is a member of the polyethylene glycol derivatives that also include polysorbate 20, 40, 60, and 80.

MCT oils are triglycerides whose fatty acids have an aliphatic tail of 6-12 carbon atoms.

Room temperature and pressure means from 20 to 27 degrees Celsius at approximatelyioo kPa.

Solid means a substance that is not a liquid or a gas at room temperature and pressure. A solid substance may have one of a variety of forms, including a monolithic solid, a powder, a gel, or a paste.

Liquid means a substance that is not a solid or a gas at room temperature and pressure. A liquid is an incompressible substance that flows to take on the shape of its container.

Solutions lack an identifiable interface between the solubilized molecules and the solvent. In solutions, the solubilized molecules are in direct contact with the solvent.

Solubilized means that the oil-soluble species to be delivered is in the solution of the droplet. When solubilized, dissociation (thus, liquid separation or solid formation) of the oil-soluble species does not result in droplet average particle diameters greater than 200 nm as determined by DLS and discussed further below, or by the formation of precipitated crystals of the oil-soluble species visible with the naked eye. Thus, if either average particle diameters greater than 200 nm or precipitated crystals visible to the naked eye form, the oil-soluble species is not solubilized in the solution of the droplet. If an oil-soluble species is not solubilized in the solution, it is insoluble in the solution. In many respects, solubility may be thought of as a concentration dependent continuum. For example, the following descriptive terms may be used to express solubility of a solute in a solvent (grams solid/mL of solvent) at 25 degrees Celsius:

| Descriptive Level | Parts solvent per 1 part of solute |
|---|---|
| Very Soluble | Less than 1 |
| Freely Soluble | From 1 to 10 |
| Soluble | From 10 to 30 |
| Sparingly Soluble | From 30 to 100 |
| Slightly Soluble | From 100 to 1000 |
| Very Slightly Soluble | From 1000 to 10,000 |
| Insoluble | More than 10,000 |

Dissociation occurs when a previously solubilized solid or liquid leaves a solution and is no longer in direct contact with a solvent of the solution. Dissociation of solids from the solvent occurs through recrystallization, precipitation, and the like. Dissociation of liquids from the solvent occurs through separation and the formation of a visible meniscus between the solvent and the dissociated liquid.

A shelf-stable microemulsion may be determined in one of two ways. One way to establish that a microemulsion stored in a sealed container substantially excluding air and moisture is shelf-stable is when dissociation of a solid does not occur and the oil phase droplets in the water do not change in average diameter by more than +/−20% at about 25° C. for a time period of at least 3 months to 2 years, preferably for a time period of at least 6 months to 2 years, and more preferably, for a time period of at least 1 year to 2 years. Another way to establish that a microemulsion is shelf-stable is when dissociation of a solid does not occur and the oil phase droplets in the water do not separate into a visibly distinct phase with a visible meniscus when stored in a sealed container substantially excluding air and moisture at about 25° C. for a time period of at least 6 months to 2 years, and more preferably, for a time period of at least 1 year to 2 years. Either type of dissociation means that the microemulsion is not shelf-stable.

Emulsions are mixtures of two or more liquids that do not solubilize. Thus, one of the liquids carries droplets of the second liquid. The droplets of the second liquid may be said to be dispersed in a continuous phase of the first liquid. An interface, separation, or boundary layer exists between the two liquids, thus between the carrier liquid (continuous phase) and the droplets of the second liquid. Emulsions may be macroemulsions, pseudo-emulsions, microemulsions, or nanoemulsions. The primary difference between the emulsion types is the size (average diameter) of the droplets dispersed in the continuous phase and whether the droplets are thermodynamically stable in the continuous phase. Macroemulsions and pseudo-emulsions have average droplet diameters from 1 to 20 micrometers. Microemulsions and nanoemulsions have smaller average droplet diameters in the continuous phase than macroemulsions and pseudo-emulsions. Microemulsions are thermodynamically stable while nanoemulsions are not even though their average droplet diameters may overlap in size.

Macroemulsions are thermodynamically unstable but kinetically stable dispersions of oil in water, with oil being defined as any water-insoluble liquid. By thermodynamically unstable it is meant that once created, the macroemulsion is always reverting to the original, immiscible state of the oil and water constituents (demulsification), but this break down is slow enough (thus, kinetically “stable”) that the macroemulsion may be considered stable from an intended use practicality perspective. Macroemulsions scatter light effectively and therefore appear milky, because their droplets are greater in diameter than the wavelength of visible light. The IUPAC definition of a macroemulsion is an “emulsion in which the particles of the dispersed phase have diameters from approximately 1 to 100 micrometers. Macro-emulsions comprise large droplets and thus are ‘unstable’ in the sense that the droplets sediment or float, depending on the densities of the dispersed phase and dispersion medium.”

Pseudo-emulsions are dispersions of oil in water, with oil being defined as any water-insoluble liquid, including tiny (micronized) solid granules that are not fully solubilized in the oil droplets. The term “pseudo-emulsion” is used as these mixtures are not true emulsions as the solid granules are not fully solubilized into the droplets. The droplets of a pseudo-emulsion generally have an average droplet diameter of 1 to 20 micrometers, thus being a “solid granule modified macroemulsion”.

Microemulsions are thermodynamically stable dispersions of oil in water, with oil being defined as any water-insoluble liquid. Microemulsion are made by simple mixing of the components. Thus, microemulsions spontaneously form or “self-assemble” and do not require high shear forces. Unlike macroemulsions, microemulsions do not substantially scatter light. The IUPAC definition of a microemulsion is a “dispersion made of water, oil, and surfactant(s) that is an isotropic and thermodynamically stable system with dispersed domain diameter varying approximately from 1 to 100 nm, usually 10 to 50 nm.” Thus, the droplets of a microemulsion are approximately three orders of magnitude smaller than the droplets of a macroemulsion and are thermodynamically stable.

Nanoemulsions have average droplet diameters from 10 to 125 nanometers, thus being at least an order of magnitude smaller in average droplet diameters than macro- and pseudo-emulsions. Nanoemulsions are made with mechanical, high-energy forces—such as provided by high-pressure homogenization, high-shear mixers, such as bead mills and rotor-stator mixers, and ultrasonic mixers. While the average droplet diameter of nanoemulsions and microemulsions formally overlap, in practice, the average droplet diameter of nanoemulsions is or become larger than those of microemulsions, as lacking the thermodynamic stability of microemulsions, the average droplet diameter of nanoemulsions is forever increasing. It is possible to apply the high energy forces required to form a nanoemulsion to a composition capable of forming a thermodynamically stable microemulsion, however, this will result in a microemulsion as the composition would have “self-assembled” without the high energy forces to form the microemulsion.

Droplets or liquid particles are formed by the hydrophobic “oil” phase of a microemulsion and are carried by the hydrophilic continuous phase. The exterior of the droplets is defined by a boundary layer that surrounds the volume of each liquid droplet. The boundary layer of a droplet defines the exterior surface of the droplets forming the dispersed oil phase of the microemulsion. The continuous phase of the microemulsion resides exterior to the boundary layer of the droplets, and thus, carries the droplets.

Continuous phase means the portion of a microemulsion that carries the droplets that include the substance to be delivered. For example, the modified oil-in-water microemulsions (non-polar droplets in polar continuous phase) addressed herein have oil droplets including the oil-soluble species to be delivered carried in a polar, “water” continuous phase. While the words “water” and “oil” are used, the “water” can be any liquid that is more polar than the “oil” (such as a polar oil), and the “oil” can be any liquid that is less polar than the “water. Thus, the terms “polar continuous phase” and “water continuous phase” are synonymous, unless water is specifically being discussed as one of the microemulsion components.

Average droplet diameter is determined by dynamic light scattering, sometimes referred to a photon correlation spectroscopy. The determination is made between 20 and 25 degrees Celsius. One example of an instrument suitable for average droplet diameter determination is a Nicomp 380 ZLS particle sizer as available from Particle Sizing Systems, Port Richey, FL. DLS can determine the diameter of droplets in a liquid by measuring the intensity of light scattered from the droplets to a detector over time. As the droplets move due to Brownian motion the light scattered from two or more droplets constructively or destructively interferes at the detector. By calculating the autocorrelation function of the light intensity and assuming a droplet distribution, it is possible to determine the sizes of droplets from 1 nanometer (nm) to 5 micrometers (um). The instrument is also capable of measuring the Zeta potential of droplets.

A visually clear microemulsion has an average droplet diameter of 200 nm and less and lacks precipitated solid crystals visible to the naked eye.

A transparent microemulsion or nanoemulsion has an average droplet diameter from 10 to 100 nanometers. Thus, a transparent microemulsion or nanoemulsion is visually clear, but a visually clear microemulsion or nanoemulsion may or may not also be transparent.

Ingestible means capable of being ingested through the mouth by a living mammal while edible means fit to be eaten, thus in contrast to being unpalatable or poisonous. Edible also means that the composition has less than the permitted amount of viable aerobic microorganisms and meets the American Herbal Products Association (AHPA) guidelines for metals, adulterants, toxins, residual solvents, and pesticides.

Subject refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. Thus, the term “subject” may be used in reference, for example, to a mammalian subject, such as a male or female human.

An “effective amount” or “effective bloodstream concentration” means an amount of deliverable effective to provide the desired result or health benefit such that a desired result is achieved. The terms also refer to the amount of deliverable sufficient to elicit the biological response of a cell, tissue, system, or subject desired by a health care professional.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these ranges may independently be included in the ranges, and are also encompassed within the invention, subject to any specifically excluded limit in the stated range. Where the stated range includes one or both of the upper and lower limits, ranges excluding either or both of those included limits are also included in the invention.

Unless otherwise indicated, all numbers expressing quantities of ingredients, ratios, and the like used in the specification and claims are to be understood as indicating both the exact values as shown and as being modified by the term “about”. Thus, unless indicated to the contrary, the numerical values of the specification and claims are approximations that may vary depending on the desired properties sought to be obtained and the margin of error in determining the values. At the very least, and not as an attempt to limit the application of the doctrine of equivalents to the scope of the claims, each numerical parameter should at least be construed considering the margin of error, the number of reported significant digits, and by applying ordinary rounding techniques.

The terms “a”, “an”, and “the” used in the specification claims are to be construed to cover both the singular and the plural, unless otherwise indicated or contradicted by context. No language in the specification should be construed as indicating any non-claimed element to be essential to the practice of the invention.

While various aspects of the invention are described, it will be apparent to those of ordinary skill in the art that other aspects and implementations are possible within the scope of the invention. Accordingly, the invention is not to be restricted except in light of the attached claims and their equivalents.

1. A gummy confection composition comprising:
a modified oil-in-water microemulsion including a modified oil phase, a modified polar continuous phase, and an oil-soluble species,
where the oil-soluble species is solubilized in the modified oil phase, the modified oil phase comprising a phospholipid, a polyethylene glycol derivative, an oil, and an alcohol, and
where the modified polar continuous phase comprises a sugar or sugar alcohol and water; and
a gummy base,
where the gummy base includes solid sugar, liquid syrup, gelling agent, an edible acid, and gummy base water,
where prior to incorporation into the gummy base the modified oil-in-water microemulsion is visually clear and shelf-stable, and
where the composition is ingestible and edible.

2. The composition of claim 1, where a weight ratio of the modified oil-in-water microemulsion to the gummy base water is from 1:2 to 1:5±10%.

3. The composition of claim 1, where the solid sugar is chosen from cane sugar, erythritol, xylitol, coconut palm sugar, tagatose, and combinations thereof.

4. The composition of claim 1, where the liquid syrup is chosen from tapioca syrup, potato syrup, corn syrup, rice syrup, wheat tapioca syrup, and combinations thereof.

5. The composition of claim 1, where the gelling agent is chosen from pectin, gelatin, and combinations thereof.

6. The composition of claim 1, where the edible acid is chosen from citric acid, malic acid, fumaric, acid, tartaric acid, and combinations thereof.

7. The composition of claim 1 having a pH from 3.3 to 3.5.

8. The composition of claim 1, where the water from the modified oil-in-water microemulsion comprises 0.2% to 6% of the composition by weight.

9. The composition of claim 1, the modified oil-in-water microemulsion configured to provide uptake of the oil-soluble species to the bloodstream of a subject at an effective bloodstream concentration through the oral and gastric mucosa of the subject.

10. The composition of claim 1, where the modified oil phase is configured to better solubilize the oil-soluble species than the oil alone.

11. The composition of claim 1, where the modified oil phase is dispersed in the modified polar continuous phase prior to incorporation of the modified oil-in-water microemulsion into the gummy base.

12. The composition of claim 11, where droplets of the modified oil phase have an average droplet diameter of 7 to 30 nanometers.

13. The composition of claim 1, where the oil-soluble species is chosen from Cannabis extracts, terpenes, oil-soluble vitamins, and combinations thereof.

14. The composition of claim 13, the Cannabis extracts chosen from cannabidiol, tetrahydrocannabinol, other cannabinoids, and combinations thereof.

15. The composition of claim 13, the terpenes chosen from limonene, pinene, linalool, beta-caryophyllene, retinol, phytol, myrcene, humulene, ocimene, terpinolene, geraniol, geranylgeraniol, and combinations thereof.

16. The composition of claim 13, the oil-soluble vitamins chosen from Vitamin A, Vitamin D, Vitamin E, Vitamins K1, Vitamin K2, carotenoids, and combinations thereof.

17. The composition of claim 1, further comprising an alcohol-soluble deliverable in the modified oil phase, the alcohol-soluble deliverable chosen from a plant sterol, a polyphenol, an anti-microbial, and combinations thereof.

18. The composition of claim 1, further comprising a water-soluble deliverable in at least one of the gummy base, the modified polar continuous phase, and combinations thereof.

19. The composition of claim 18, where the water-soluble deliverable is in the gummy base and is chosen from theanine, Vitamin C, B Vitamins, trimethylglycine, gamma-aminobutyric acid (GABA), theanine, elderberry, zinc citrate, skull cap, and combinations thereof.

20. The composition of claim 1, where the phospholipid is a glycerophospholipid isolated from lecithin.

21. The composition of claim 20, where the phospholipid is chosen from phosphatidylcholine, phosphatidylethanolamine, phosphatidylinositol, ceramide phosphoryl ethanolamine, ceramide phosphoryl choline (SPH), and combinations thereof.

22. The composition of claim 20, where the phospholipid is chosen from phosphatidylcholine, phosphatidylethanolamine, and combinations thereof.

23. The composition of claim 20, where the phospholipid is at least 80% by weight phosphatidylcholine.

24. The composition of claim 1, where the polyethylene glycol derivative is chosen from polyethylene glycol modified vitamin E, polysorbate 40, polysorbate 60, polysorbate 80, and combinations thereof.

25. The composition of claim 24, where the polyethylene glycol derivative is chosen from tocopheryl polyethylene glycol succinate 1000, polysorbate 60, polysorbate 80, and combinations thereof.

26. The composition of claim 1, where the polyethylene glycol derivative is tocopheryl polyethylene glycol succinate 1000.

27. The composition of claim 1, the oil chosen from a medium chain triglyceride, a citrus oil, and combinations thereof.

28. The composition of claim 27, the medium chain triglyceride chosen from caproic acid (hexanoic acid), caprylic acid (octanoic acid), capric acid (decanoic acid), lauric acid (dodecanoic acid), and combinations thereof.

29. The composition of claim 27, the medium chain triglyceride chosen from caprylic acid, capric acid, and combinations thereof.

30. The composition of claim 27, the citrus oil chosen from orange oil, lemon oil, and combinations thereof.

31. The composition of claim 1, where the alcohol is 95% ethanol by weight.

32. The composition of claim 1, the sugar or sugar alcohol chosen from sucrose, cane sugar, pure maple syrup, glycerol, and combinations thereof.

33. The composition of claim 1, the sugar or sugar alcohol chosen from pure maple syrup, glycerol, and combinations thereof.

34. The composition of claim 1, where the sugar or sugar alcohol is glycerol.

35. The composition of claim 1, where prior to incorporation into the gummy base the oil-soluble species comprises from 1% to 5% of the modified oil-in-water microemulsion by weight.

36. The composition of claim 1, where prior to incorporation into the gummy base the oil-soluble species comprises from 2% to 4% of the modified oil-in-water microemulsion by weight.

37. The composition of claim 1, where prior to incorporation into the gummy base the ratio of the phospholipid, to the oil, to the polyethylene glycol derivative, to the alcohol, to the sugar or sugar alcohol, and to the water is 1:2:0.6-3.3:4:7.5-9:2-3.5±20% by weight for the modified oil-in-water microemulsion.

38. The composition of claim 1, where prior to incorporation into the gummy base the ratio of the phospholipid, to the oil, to the polyethylene glycol derivative, to the alcohol, to the sugar or sugar alcohol, and to the water is 1:2:0.6-3.3:4:7.5-9:2-3.5±10% by weight for the modified oil-in-water microemulsion.

39. The composition of claim 1, the modified oil phase further comprising an oil, where the ratio of the oil to the oil-soluble species is 1:0.05 to 0.3±10% by weight.

40. The composition of claim 1, the modified oil phase further comprising an oil, where the ratio of the oil to the oil-soluble species is 1:0.05 to 0.3±5% by weight.

41. The composition of claim 1, where prior to incorporation into the gummy base the phospholipid comprises from 2% to 10% of the modified oil-in-water microemulsion by weight.

42. The composition of claim 1, where prior to incorporation into the gummy base the polyethylene glycol derivative comprises from 5% to 15% of the modified oil-in-water microemulsion by weight.

43. The composition of claim 1, where the ratio of the phospholipid to the polyethylene glycol derivative is 1:0.4 to 1:4 by weight.

44. The composition of claim 1, where the ratio of the phospholipid to the polyethylene glycol derivative is 1:1.6 to 1:4 by weight.

45. The composition of claim 1, where prior to incorporation into the gummy base the oil comprises from 5% to 15% of the modified oil-in-water microemulsion by weight.

46. The composition of claim 1, where prior to incorporation into the gummy base the alcohol comprises from 5% to 25% of the modified oil-in-water microemulsion by weight.

47. The composition of claim 1, where the oil to the alcohol ratio is 1:1.5 to 1:4 by weight.

48. The composition of claim 1, where prior to incorporation into the gummy base the sugar or sugar alcohol comprises from 30% to 55% of the modified oil-in-water microemulsion by weight.

49. The composition of claim 1, where prior to incorporation into the gummy base the sugar or sugar alcohol comprises from 30% to 45% of the modified oil-in-water microemulsion by weight.

50. The composition of claim 1, where prior to incorporation into the gummy base the water comprises from >10% to 25% of the modified oil-in-water microemulsion by weight.

51. The composition of claim 1, where prior to incorporation into the gummy base the water comprises from 11% to 14% of the modified oil-in-water microemulsion by weight.

52. The composition of claim 1, the composition configured to provide a human subject a from 4 to 6 ng/mL blood concentration of the oil-soluble species within 20-minutes of the human subject chewing and ingesting the composition including 12 mg of the oil-soluble species.

53. The composition of claim 1, the composition configured to provide a human subject with a Cmax blood concentration of the oil-soluble species within 30 to 50 minutes of the human subject chewing and ingesting the composition.

54. The composition of claim 1, the composition configured to provide a human subject from 60 to 90 times more oil-soluble species in the bloodstream within 40-minutes of the human subject chewing and ingesting the composition than when the oil-soluble species is delivered to the human subject orally in an oil blend.

55. The composition of claim 1, the composition configured to provide a human subject from 2 to 4 times more oil-soluble species in the bloodstream within 20-minutes of the human subject chewing and ingesting the composition than when the oil-soluble species is delivered to the human subject through chewing and ingesting a conventional gummy confection lacking the modified oil-in-water microemulsion.

56. The composition of claim 1, the composition configured to provide a human subject approximately twice as much oil-soluble species in the bloodstream within 90-minutes of the human subject chewing and ingesting the composition than when the oil-soluble species is delivered to the human subject through chewing and ingesting a conventional gummy confection lacking the modified oil-in-water microemulsion.

57. A method of making a gummy confection composition, the method comprising:
combining a phospholipid, a polyethylene glycol derivative, an oil, and an alcohol to form an alcohol-lipid mixture;
combining a sugar or sugar alcohol and water to form a modified polar continuous phase;
combining an oil-soluble species with the alcohol-lipid mixture and the modified polar continuous phase at atmospheric pressure to form a modified oil-in-water microemulsion;
combining a first component of a solid sugar, a gelling agent, and additional water to form a first a first gummy base component;
combining a second component of the solid sugar, a liquid syrup, and additional water to form a second gummy base component;
combining the first gummy base component, the second gummy base component, and the modified oil-in-water microemulsion to form a combination;
transferring the combination to a mold; and
forming the gummy confection composition in the mold upon cooling.

58.-122. (canceled)
